SEQUENCE LISTING (21 sequences)

SEQ ID NO: 1 - protein, 98 aa, Artificial Sequence
  Human CD74 invariant chain fragment
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLP

SEQ ID NO: 2 - protein, 286 aa, Artificial Sequence
  Chicken CD 74 invariant chain fragment
MAEEQRDLISSDGSSGVLPIGNSERSSLGRRTALSALSILVALLIAGQAVTIYYVYQQSGQISKLTKTSQTLKLESLQRKMPIGTQPANKMSMSTMNMPMAMKVLPLAPSVGDMPMEAMKPRSNKTEDQIRHLLLKSDPRKTFPDLKDDMLGNLKRLKKTMSAMDWQDFETWMHKWLLFEMAKGPKMEEQNTIPAEKVQTKCQAEASFGGVHPGRFRPECDENGDYLPKQCYASTGYCWCCYKNGTRIEGTATRGQLDCSAPAPTQPPSAEPEEVIFSGVDMVKAK

SEQ ID NO: 3 - protein, 285 aa, Artificial Sequence
  Quail CD74 invariant chain fragment.
MAEEQRDLISSDGSSGVLPIGNNERSAVGRRTVLSAMSILVALLIAGQAVTIYYVYQQSGQISKLTKTSQTLKLEALQKKLPMSAQPANKMSMSMMNMPMAMKVLPLAPSVGDMPMEAVRPRSNKTEDQVRHLLLKADPRKTFPELKEDLLGNLKRLKKAMNAMDWQDFEAWMHKWLLFEMAKGPKGEEQNAIPAEKVQTKCQAEASFGGVHPGRFRPECDENGDYLPKQCLASTGYCWCCYKNGTRIEGTATRGELDCSVAAPAPTPTEPEEMIFSGVDMLKAK

SEQ ID NO: 4 - protein, 200 aa, Artificial Sequence
  Trout CD 74 invariant chain fragment
MSDPQRQPLIGASSEQTAINVGTTAEGSNKRAFKIAGFTLLACLLIAGQALTAYFVLGQKNDIKDLQEESKSLKNELSQGRAAAVPMKLHVPMNTKPLLIDESVDEGTSTQGPKEGSGSPTQCQLESTGLKPVSIESFRPQCDEQGNYLLQQCLNDKPLCWCVDASGKQLSGTVTSGPARCGATSALNHVMAIPDVMPSN

SEQ ID NO: 5 - protein, 198 aa, Artificial Sequence
  Zebra fish CD 74 invariant chain fragment
MSSEGNETPLISDQSSVNMGPQPRNKNQALKVAGVTLLAGILIAGQAFTAYMAYSHKEQLNTLERRGDRLHEISRKSAMRAPLKMHLPMSSLALKYEDDTKENKDSPPSSPKPEPKVLTQCQKEAAGEVKSLLPSFKPRCNENGDYLSQQCWEQTDFCWCVDKNGVEIPDTLKEGPAQCWALNSADIVNEPLLRKNGE

SEQ ID NO: 6 - protein, 237 aa, Artificial Sequence
  zebra fish CD 74 invariant chain fragment
MEPDHQNESLIQRVPSAETILGRGDARNSNTKALKITGLTVLACLLLAGQALTAYMVWGQKQHINALTSGQEKLKAELTRKMSAGAPKAMSLPMNSMPLLKDYSDPTSDQTAVKSKSVPLTKLRPIYSNQREGSGQVDGSRLMPKMMHLPMRSMPLLVNADEDVKSSPESVVELETKCKLESEREVRPGFFKPACDEEGNYMPMQCWHSTGYCWCVTKDGTEIEGTRIRGRPQCESV

SEQ ID NO: 7 - protein, 232 aa, Artificial Sequence
  Carp CD74 invariant chain fragment
MDEHQNESLIQRVPSEETVLSRGPTRNSNGKALKVTGLTVLACLLLAGQALTAYLVWGQKEHISALTSGQEKLKTELTRKVSAGPPKAMHLPMKSMPLLKDFSDETSDQTSDKKKSGSLVKLHPVFTNQREGSGQLDGARMMHLPMKSMPLLVDADEEVKSSPESAVEVETKCKLESEKQVRPGFFRPQCDEQGNYLPMQCWHSTGYCWCVDKDGNEIPDTRTRGRPKCDSE

SEQ ID NO: 8 - protein, 293 aa, Artificial Sequence
  Frog CD 74 invariant chain fragment
MAEESQNLVPEHVPGQSVVDVGNGERRMSCNKGSLVTALTVLVAVLVAGQAVMAFFITQQNSRIQDLDRSTKNLQLKAMMKELPGSPPVPSKQKMRTFNIPMALKLYDGSEMNMNDLEQMAQTGNKMEDAAKYMLLRGNPLRKYPSLNGTILENLRELKKTLTDQEWMTFDAWMQQWYLFFLVQNTEKPAEPLPSTKNTAVTGAPLMTECQTLSRIHTMTGTYKPQCEQNGDFKPLQCWPSTGFCWCVYHNGTEIPETRTRSRLDCSSIMQPEDPMFLESITSSDADFGPFDM

SEQ ID NO: 9 - protein, 178 aa, Artificial Sequence
  Grouper CD 74 invariant chain fragment
MAEPAEGAPLAAGSLSGSEEVLVLPAAPRGGSNSRALKIAGLTTLACLLLASQVFTAYMMFDQKQQIHSLQRNSDKLGRQLTRSSQAVAPVRMHMPMNSLPLLKDFDADANTKPKKTPLTKLKEAVVNVEAQLKELLQEDSQLPEFNETFLANLQGMKQHVSETDWKSFESWMRYWLI

SEQ ID NO: 10 - protein, 243 aa, Artificial Sequence
  Shark CD 74 invariant chain fragment
MSADEQQNALLNNSQQDIASQSSVEARTTVTGQSPTCSKSLLWGGVTVLAAMLIAGQVASVVFLVKQQSTITHLQQTTSLIQQKYTSNRNRFPPKPMIHVKPMILDMPMAYIDPNAERPKIPKPTPAKPLTVLEQVEELLKKDNATKEIPEFNSTFSANLDLLRDSMTESEWQDFETWLRNWLFFQLIQEKKSAPTTSAPRFDPIPEPEPQPRERKIFSSVAMKSMMVSLPEPAKAPVSKKAN

SEQ ID NO: 11 - protein, 292 aa, Artificial Sequence
  Mandarin fish CD 74 invariant chain
MAEESQNLVPEHVPGQSVVDVGNGERRMSCNKGSLVTALTVLVAVLVAGQAVMAFFITQQNSRIQDLDRSTKNLQLKAMMKELPGSPPVPSKQKMRTFNIPMALKLYDGSEMNMNDLEQMAQTGNKMEDAAKYMLLRGNPLRKYPSLNGTILENLRELKKTLTDQEWMTFDAWMQQWYLFFLVQNTEKPAEPLPSTKNTAVTGAPLMTECQTLSRIHTMTGTYKPQCEQNGDFKPLQCWPSTGFCWCVYHNGTEIPETRTRSRLDCSSIMQPEDPMFLESITSSDADFGPFD

SEQ ID NO: 12 - protein, 294 aa, Artificial Sequence
  Mallard CD 74 invariant chain fragment
MVWGQWGPQGSSIGHWEGIWQCVTGSPPAVSRRSAFGRRAALSTLSILVALLIAGQAVTIYFVYQQSGQISKLTRTSQNLQLEALQRKLPKSSKSTGNMKMSMVNTPLAMRVLPLAPSLDDTPVKDMGPPSNKTEDQVRHLLLQADPKKMFPELKDSLLGNLKSLKKTMTDADWKSFESWMHKWLLFEMAKSPKPDERKAVPAEKVQTKCQAEANFGGVHPGRFRPECDENGDYLPKQCHAGTGYCWCCYKNGTKIEGTATRGELDCSGRWRGEPDEMIFSGVDMLKLGAEKAK

SEQ ID NO: 13 - protein, 232 aa, Artificial Sequence
  Human CD 74 invariant chain
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPM

SEQ ID NO: 14 - protein, 26 aa, Artificial Sequence
  Human CD74 invariant chain truncated
GALYTGFSILVTLLLAGQATTAYFLY

SEQ ID NO: 15 - protein, 92 aa, Artificial Sequence
  Human CD74 invariant chain fragment
MHRRRSRSCREDQKPVMDDQRDLISNNEQLPMLGRRPGAPESKCSRGALYTGFSILVTLLLAGQATTAYFLYQQQGRLDKLTVTSQNLQLEN

SEQ ID NO: 16 - protein, 72 aa, Artificial Sequence
  Human CD74 invariant chain fragment
MHRRRSRSCREDQKPVMDDQRDLISNNEQLPMLGRRPGAPESKCSRGALYTGFSILVTLLLAGQATTAYFLY

SEQ ID NO: 17 - protein, 57 aa, Artificial Sequence
  Trout CD 74 invariant chain truncated
MSDPQRQPLIGASSEQTAINVGTTAEGSNKRAFKIAGFTLLACLLIAGQALTAYFVL

SEQ ID NO: 18 - protein, 65 aa, Artificial Sequence
  Shark CD 74 invariant chain truncated
MSADEQQNALLNNSQQDIASQSSVEARTTVTGQSPTCSKSLLWGGVTVLAAMLIAGQVASVVFLV

SEQ ID NO: 19 - protein, 56 aa, Artificial Sequence
  Chicked CD 74 invariant chain truncated
MAEEQRDLISSDGSSGVLPIGNSERSSLGRRTALSALSILVALLIAGQAVTIYYVY

SEQ ID NO: 20 - protein, 54 aa, Artificial Sequence
  Zebra fish CD 74 invariant chain trucated
MSSEGNETPLISDQSSVNMGPQPRNKNQALKVAGVTLLAGILIAGQAFTAYMAY

SEQ ID NO: 21 - protein, 58 aa, Artificial Sequence
  Frog CD 74 invariant chain fragment
MAEESQNLVPEHVPGQSVVDVGNGERRMSCNKGSLVTALTVLVAVLVAGQAVMAFFIT

(19) United States
(12) Patent Application Publication
(10) Pub. No.: US 2016/0304582 A1
(43) Pub. Date: Oct. 20, 2016

(54) MOLECULAR ADJUVANT

(71) Applicant: ISIS INNOVATION LIMITED, Oxford Oxforedshire (GB)

(72) Inventors: Adrian Vivian Sinton Hill, Oxford (GB); Alexandra J. Spencer, Oxford (GB); Benedict R. Halbroth, Oxford (GB); Matthew G. Cottingham, Oxford (GB)

(73) Assignee: ISIS INNOVATION LIMITED, Oxford (GB)

(21) Appl. No.: 15/101,282

(22) PCT Filed: Dec. 3, 2014
(86) PCT No.: PCT/GB2014/053596

§ 371 Date: Jun. 2, 2016

(30) Foreign Application Priority Data
Dec. 4, 2013 (GB) 1321384.8

(51) Int. Cl. C07K 14/74; A61K 39/39; C12N 7/00; C07K 14/445; A61K 39/015

(52) U.S. Cl. CPC C07K 14/70539; C07K 14/445; A61K 39/015; C12N 7/00; A61K 39/39; C07K 2319/00; A61K 2039/53

(57) ABSTRACT

The invention relates to the use of a molecular adjuvant to generate an improved immune response in a host. Over recent years extensive research and development has been undertaken in the development of “vectored vaccines” which can be used as vaccine delivery systems. Vectored vaccines include DNA vectors and recombinant viral and bacterial vectors, which are engineered to express an antigen of interest. The invention provides a nucleic acid construct encoding a protein fusion between an antigen and an invariant chain molecule.

This invention relates to the use of a molecular adjuvant to generate an improved immune response in a host.

Over recent years extensive research and development has been undertaken in the development of “vectored vaccines” which can be used as vaccine delivery systems. Vectored vaccines include DNA vectors and recombinant viral and bacterial vectors, which are engineered to express an antigen of interest.

Ideally recombinant viral and bacterial vectored vaccines are unable to replicate in the cells of the vaccine recipient, thereby enhancing safety of the vaccines. Viruses which may be used in the production of such vectored vaccines include human and non-human adenoviruses, vaccinia, modified vaccinia Ankara (MVA), other poxviruses, adenovirus associated viruses, flaviviruses, herpes viruses, alpha viruses and other suitable viruses. Poxviruses have been proposed as good candidates for vectored vaccines as they show high species specificity; for example, avipox virus is unable to replicate in mammalian cells (Paoletti 1996 PNAS USA 93, 11349-11353). Protective T-cell responses induced by recombinant vaccinia viruses in small animals were first reported in the 1980s (Panicali and Paoletti 1982 PNAS USA 79, 4927-4931; Smith et al 1983 Nature 302, 490-495). Subsequently the highly attenuated recombinant vaccinia virus MVA (modified vaccinia virus Ankara) (Sutter and Moss 1992 PNAS USA 89, 10847-10851) and NYVAC (New York vaccinia) (Tartaglia et al 1992 Virology 188, 217-232) have been shown to have good immunogenicity. Due to an acquired replication defect MVA does not replicate in human cells, but is able to express recombinant genes. Similarly, NYVAC has been molecularly attenuated to prevent replication in human cells.

The aim of vectored vaccines is to activate cell-mediated or antibody-mediated immunity in a host organism against an antigen of interest. Preferably the cell-mediated immunity includes stimulation of a T cell response. This may be evidenced by a CD4+ and/or a CD8+ T cell response in a host organism following administration of the vectored vaccine. T cells are critical components of the immune system, and are involved in the control of intracellular pathogens. An intracellular stage is a feature of many pathogens including Plasmodium spp, M. tuberculosis, Leishmania and HIV. The T cell role extends beyond the control of infectious disease, for example some tumours express tumour-associated antigens which can be controlled by T cells targeting them. Vaccines designed to elicit T cell based protection against diseases, such as cancer and tumours, are under development (Hill, A. V 2006 Nat Rev Immunol, 6(1), 21-32; Sander, C and McShane, H 2007 Clin Exp Immunol, 147(3), 401-11; Johnston, M. I. and Fauci, A. S 2007 N Engl J Med, 356(20) 2073-81; Kedzierski, L et al 2006 Parasitology, 133 Suppl, S87-112; Xue, S. A and Stauss, H. J 2007 Cell Mol Immunol, 4(3), 173-84), but limited immunogenicity and protective efficacy of the vaccines remain a serious concern.

Thus, whilst vectored vaccines offer a good basis for developing new vaccines, the immune response elicited by such vaccines when administered to an organism, typically a human, is often not sufficiently strong to provide protection against infection and/or disease related to the antigen encoded by the vector. Where the antigen is from a pathogen, the vectored vaccine may be intended to confer protection from infection and/or disease caused by the pathogen from which the antigen of interest is derived. Alternatively, the antigen may be derived from a particular cancer or disease and the vectored vaccine may be intended to confer protection or to treat that particular cancer or disease in the host organism.

A known method to enhance the immune response of an organism to an antigen in a vaccine is to use one or more adjuvants (or immune potentiators). Wherein the adjuvant increases the strength and/or duration of an immune response to an antigen relative to that elicited by the antigen alone.

Known adjuvant compositions include oil emulsions (Freund's adjuvant), oil based compounds (e.g. MF59, ISA51, ISA720), saponins, aluminium or calcium salts (i.e. Alum), non-ionic block polymer surfactants, lipopolysaccharides (LPS), attenuated or killed mycobacteria, tetanus toxoid, monophosphoryl lipid A, imiquimod, resiquimod, polyl:C, CpG containing oligonucleotides, lipoproteins and others.

Many adjuvants produce undesirable side effects in humans such as inflammation at the site of injection, these side effects can limit their use and efficacy, and thus there is a need for alternative, and improved, adjuvants.

One approach to augment vaccine-induced T cell responses is genetic fusion of the antigen of interest to CD74, the MHC class II invariant chain (Ii). The now well-known, canonical role of Ii in the MHC class II antigen presentation pathway led several researchers to exploit genetic fusion of antigens to Ii as a means of enhancing antigen presentation to CD4⁺ T cells. An unexpected, additional effect on CD8⁺ T cell induction, using immunization with a lentiviral vector expressing ovalbumin fused to Ii (li-OVA) has also been described (Rowe et al 2006 Mol Ther 13(2) 310-9). Subsequently, four reports from the University of Copenhagen have documented enhanced induction of CD8⁺ T cell responses by human adenovirus 5 (HAdV-5) and plasmid DNA vectors expressing li-fused antigens.

Vaccination with the glycoprotein of lymphocytic choriomeningitis virus (LCMV-GP), expressed by a HAdV-5 vector (Hoist et al 2008 J Immunol, 180(5), 3339-46) or a DNA plasmid (Grujic et al 2009 J Gen Virol 90(Pt 2), 414-22), was shown to elicit higher frequencies of antigen-specific IFN-γ⁺ CD8⁺ T cells and enhanced in vivo proliferation of adoptively transferred CD8⁺ T cells when fused to li. HAdV-5 vectored vaccines encoding the li-fused antigen conferred improved protection in an LCMV challenge model and in a tumour challenge model using melanoma cells expressing LCMV-GP (Sorensen et al 2009 Eur J Immunol 39(10), 2725-36), consistent with prior independent results using lentivirally-delivered li-OVA and EG7-OVA tumour challenge cells (Rowe et al 2006 Mol Ther 13(2), 310-9). Similarly, fusion of the NS3 protein of hepatitis C virus to li accelerated and augmented IFN-γ⁺ CD8⁺ T cell responses following vaccination with a HAdV-5 vector encoding this antigen, with no significant difference in cellular phenotype, as assessed by multi-parameter flow cytometry. Furthermore, the reduction in viral titre after challenge with vaccinia virus expressing NS3 was enhanced in an IFN-γ-dependent manner (Mikkelsen et al 2011 J Immunol 186(4), 2355-64).

Genetic fusion of a transgenic antigen to li may augment CD8⁺ T cell immunogenicity via enhanced antigen presentation on MHC class I. Bone-marrow derived dendritic cells transduced with vectors expressing li-fused antigen have been reported to direct greater antigen-specific CD8⁺ T cell proliferation in vitro, without any apparent difference in levels of cell surface costimulatory molecules or total MHC class I (Hoist et al 2008 J Immunol 180(5), 3339-46). Furthermore, CD4⁺ T cell help is required neither for this in vitro effect nor for enhancement of HAdV-5 vector-induced CD8⁺ T cell responses in vivo (Hoist et el 2011 J Immunol 186(7), 3997-4007). The mechanism of the effect of li on MHC class I presentation of a fused antigen remains unclear, mainly due to the large number of functional domains of li that could be responsible. Nevertheless, the available evidence supports progression of these research findings into an appropriate clinical setting, such as a liver-stage malaria vaccine known to elicit protective human CD8⁺ T cell responses against P. falciparum. /

ME-TRAP is an antigenic construct comprising full-length Plasmodium falciparum TRAP (thrombospodin related adhesion protein or sporozoite surface protein 2) fused to ME, a string of 20 malarial T- and B-cell epitopes. Heterologous prime-boost immunization of healthy adults with vectored vaccines encoding ME-TRAP delayed or prevented parasitaemia in a proportion of volunteers challenged with P. falciparum infection in three phase 2a clinical efficacy trials (Dunachie et al 2006 Infect Immun 74(10), 5933-42; Webster et al 2005 PNAS 102(13), 4836-41; McConkey et al 2003 Nat Med 9(6), 729-35). As new viral vectors have been developed and incorporated into optimized heterologous prime-boost regimens, the mean frequencies of T cells induced against ME-TRAP in humans have also increased, from tens of IFN-γ spot forming cells per million peripheral blood mononuclear cells (SFC/10⁶ PBMCs) using DNA plasmid priming and boosting with recombinant modified vaccinia virus Ankara (MVA), to hundreds of SFC/10⁶ PBMCs using priming with recombinant fowlpox virus (strain FP9) and boosting with MVA, and most recently to thousands of SFC/10⁶ PBMCs using priming with a recombinant chimpanzee adenovirus vector, ChAd63, and boosting with MVA (O′Hara et al 2012 J Infect Disease 205(5), 772-81). Since there is evidence that increased cellular immune responses against ME-TRAP correlate with increased protection against P. falciparum challenge, there is a need to improve the immunogenicity of ME-TRAP and other antigen expressing viral vectored vaccines even further.

The invention provides a nucleic acid construct encoding a protein fusion between an antigen and an invariant chain molecule. The invariant chain molecule consists of the peptide of SEQ ID NO.1 or a fragment thereof or a variant of SEQ ID NO.1 or a variant of a fragment of SEQ ID NO.1. Variants have at least 85% sequence identity with the corresponding portion of SEQ ID NO.1. The invariant chain molecule produces an enhanced CD4⁺ and/or CD8⁺ and/or antibody immune response against the antigen upon immunisation with the construct compared to the CD4⁺ and/or CD8⁺ and/or antibody immune response obtained by immunisation with a control construct encoding the antigen not fused to the invariant chain molecule.

SEQ ID No.1 is a fragment of the long isoform (isoform (b)) of the human CD74 molecule, also know as the invariant chain (Nucleic Acids Res. 1985 Dec. 20; 13(24): 8827-8841). The inventors have surprisingly found that N-terminal fragments of the invariant chain (Ii) which comprise at least the transmembrane domain thereof, provide a surprisingly effective adjuvant function when expressed as a fusion protein with an antigen of interest. Fragments encompassing the transmembrane domain and the cytoplasmic domain, and preferably including the N-terminal 16 amino acids of the long isoform of the protein are particularly efficacious.

Surprisingly, the inventors have determined that the fragments of li capable of providing the enhanced adjuvant function do not require either of the ‘KEY’ region, or the ‘CLIP’ region of the protein, previously identified as important for binding to MHC Class II. Although the KEY region of the protein is included in SEQ ID No.1 (residues 93 to 96), it's presence in the fragments used in the invention is not essential. In some embodiments, therefore, C-terminal truncations of SEQ ID NO.1 are used in which KEY is not included. In full length li, the CLIP region lies C-terminal to the fragment of SEQ ID No.1, and CLIP is therefore always excluded from the fragments used in the invention.

The fragments of li utilized in the invention therefore differ from those identified in the prior art, which has focused on experiments in which the CLIP and KEY regions have been deliberately included. The ability of the transmembrane domain to facilitate an adjuvant function in the absence of the CLIP region is a surprising finding in view of the dominant role played by CLIP in binding of li to MHC Class II.

The inventors have also surprisingly discovered that the fragments of li used in the invention provide an adjuvant effect which enhances not only the CD4⁺ response to the antigen, but also the CD8⁺ response, mediated through MHC Class I. In some embodiments only the CD8⁺ response is enhanced. Most surprisingly the CD8+ T cell response using the fragment of li may exceed that achieved when using the full length li. In some embodiments an antibody immune response is enhanced.

In some embodiments the constructs of the invention encode a fusion between an antigen and the entire polypeptide of SEQ ID NO.1. In other embodiments, N-terminal truncations, C-terminal truncations or N- and C-terminal truncations of SEQ ID NO.1 are fused to the antigen.

In some embodiments the invariant chain molecule consists of a fragment of SEQ ID NO.1 having: an N-terminus at any of positions 1 to 26 and a C-terminus at any of positions 72 to 87 of SEQ ID NO.1; an N-terminus at position 27 and a C-terminus at any of positions 72 to 75 or 77 to 87 of SEQ ID NO.1; an N-terminus at position 28 and a C-terminus at any of positions 72 to 74 or 78 to 87 of SEQ ID NO.1; an N-terminus at position 29 and a C-terminus at any of positions 72 to 73 or 79 to 87 of SEQ ID NO.1; an N-terminus at position 30 and a C-terminus at any of positions 72 or 80 to 87 of SEQ ID NO.1; an N-terminus at position 31 and a C-terminus at any of positions 81 to 87 of SEQ ID NO.1; an N-terminus at position 32 and a C-terminus at any of positions 72 or 82 to 87 of SEQ ID NO.1; an N-terminus at position 33 and a C-terminus at any of positions 72 to 73 or 79 or 83 to 87 of SEQ ID NO.1; an N-terminus at position 34 and a C-terminus at any of positions 72 to 73 or 84 to 87 of SEQ ID NO.1; an N-terminus at position 35 and a C-terminus at any of positions 72 or 85 to 87 of SEQ ID NO.1; an N-terminus at position 36 and a C-terminus at any of positions 86 to 87 of SEQ ID NO.1; an N-terminus at position 37 and a C-terminus at any of positions 72 or 87 of SEQ ID NO.1; an N-terminus at position 38 and a C-terminus at any of positions 72 to 73 or 84 of SEQ ID NO.1; an N-terminus at position 39 and a C-terminus at any of positions 72 to 74 or 78 of SEQ ID NO.1; an N-terminus at position 40 and a C-terminus at any of positions 72 to 75 or 77 of SEQ ID NO.1; an N-terminus at position 41 and a C-terminus at any of positions 72 to 76 of SEQ ID NO.1; an N-terminus at position 42 and a C-terminus at any of positions 72 to 77 of SEQ ID NO.1; an N-terminus at position 43 and a C-terminus at any of positions 72 to 78 of SEQ ID NO.1; an N-terminus at position 44 and a C-terminus at any of positions 72 to 79 or 83 of SEQ ID NO.1; an N-terminus at position 45 and a C-terminus at any of positions 72 to 80 or 82 of SEQ ID NO.1; an N-terminus at position 46 and a C-terminus at any of positions 72 to 81of SEQ ID NO.1; an N-terminus at position 47 and a C-terminus at any of positions 72 to 82 of SEQ ID NO.1; or an N-terminus at any of positions 1 to 47 and a C-terminus at any of positions 97 or 98 of SEQ ID NO.1. In some embodiments, any one or more of these fragments are excluded, and any one or more may therefore be disclaimed, leaving the remainder.

Preferred N- or C-terminal truncations of li for fusion to the antigen include amino acids 1 to 72 of SEQ ID NO.1, amino acids 47 to 98 of SEQ ID NO.1, amino acids 47 to 72 of SEQ ID NO.1, amino acids 16 to 98 of SEQ ID NO.1, and amino acids 16 to 72 of SEQ ID NO.1.

In some embodiments, the nucleic acid encoding the li molecule discussed above is replaced with nucleic acid encoding a fragment of an invariant chain from a non-human animal species, such that the nucleic acid vector encodes a protein fusion between the antigen and a non-human invariant chain molecule. In such embodiments, the fragment of the invariant chain from a non-human species comprises the transmembrane domain of the full length invariant chain from that species.

Non-human animal sources of a fragment of invariant chain include chicken, quail, trout, zebrafish, carp, frog, grouper, shark, mandarin fish or mallard, and suitable fragments from these species are encoded by the nucleic acids provided in the following SEQ ID NOs: chicken (SEQ ID NO.2), quail (SEQ ID NO.3), trout (SEQ ID NO.4), zebrafish (SEQ ID NO.5 and SEQ ID NO.6), carp (SEQ ID NO.7), frog (SEQ ID NO.8), grouper (SEQ ID NO.9), shark (SEQ ID NO.10), mandarin fish (SEQ ID NO.11), mallard (SEQ ID NO.12).

In preferred embodiments, the encoded non-human invariant chain molecule is selected from: (i) any of SEQ ID NO.s 2 to 12, or (ii) fragments of any of the sequences of SEQ ID NO.s 2 to 12 comprising the transmembrane domain thereof, or (iii) variants of the sequences in (i) or (ii) having at least 85% sequence identity therewith. The fragments and variants produce an enhanced CD4⁺ and/or CD8⁺ and/or antibody immune response against the antigen upon immunisation with the construct compared to the CD4⁺ and/or CD8⁺ and/or antibody immune response obtained by immunisation with a control construct encoding the antigen not fused to the non-human invariant chain molecule. Methods for determining immune response and for selecting an appropriate control construct are as discussed infra.

In some embodiments the sequence of the encoded li may be varied from that of SEQ ID NO.1 without abolishing the functional ability to provide an enhanced CD4⁺ and/or CD8⁺ immune response to the fused antigen. The invention therefore includes within its scope, the use of li molecules having at least 85% sequence identity to the entirety of the sequence of SEQ ID NO.1, or to the fragments of SEQ ID NO.1 noted above. Only variants (having at least 85% sequence identity) which produce an enhanced CD4⁺ and/or CD8⁺ and/or antibody immune response against the antigen upon immunisation with the construct compared to the CD4⁺ and/or CD8⁺ and/or antibody immune response obtained by immunisation with a control construct encoding the antigen but not the invariant chain molecule, are part of the invention.

In some embodiments, the human or non-human invariant chain molecule encoded by the nucleic acid of the construct is a variant of one of the sequences given in SEQ ID NOs 1 to 12, or a variant of a fragment of one of the sequences of SEQ ID NOs 1 to 12 (in each case with the proviso that the variant enhances the CD4⁺ and/or CD8⁺ and/or antibody immune response against the antigen). In preferred embodiments degree of sequence identity between the variant and the sequence provided in the corresponding SEQ ID is at least 90%, optionally at least 95%, preferably at least 96%, 97%, 98% or 99%.

The degree of sequence identity between amino acid sequences may be calculated using well known scoring matrices such as any one of BLOSUM 30, BLOSUM 40, BLOSUM 45, BLOSUM 50, BLOSUM 55, BLOSUM 60, BLOSUM 62, BLOSUM 65, BLOSUM 70, BLOSUM 75, BLOSUM 80, BLOSUM 85, and BLOSUM 90.

The skilled person is able to determine the nature and contents of a suitable control construct, but in most instances a control construct will be identical to the construct of the invention except for the absence of nucleic acid encoding the li molecule portion of the fusion protein. Alternative control constructs may include nucleic acid encoding the li molecule, for example by including a stop codon between the portion encoding the antigen and the portion encoding the li molecule. Other alternatives will be apparent to the skilled person.

Various experimental platforms are available to determine the ability of a variant li sequence or fragment of SEQ ID NO.1 to enhance the CD4⁺ and/or CD8⁺ and/or antibody immune response against the antigen (and therefore to determine whether or not it is within the scope of the invention). The skilled person is able to determine an appropriate experiment for this purpose, but may, for example, use experiments such as those presented below in the Examples. It is not necessary, and may not be desirable to perform experiments in humans to determine the adjuvant effect of a variant or fragment. It is adequate to use an animal model such as the mouse models used in the Examples herein to demonstrate an enhanced immune response to the antigen, and the invention is intended to cover the use of fragments and variants that provide an enhanced immune response in such models. The invention is also not limited to the use of fragments or variants which provide an enhanced immune response in mice. In some instances the mouse model will not be the most appropriate or predictive model and other models may be preferred, for example rat or non-human primate models, such as macaque monkeys. In other cases, in vitro determinations of the CD4⁺ and/or CD8⁺ and/or antibody immune response can be used. In such cases it is possible to determine the response in a human subject.

Demonstrating an enhanced immune response induced by fusion to a variant li sequence may be achieved by measuring increased cytokine production by ELISpot (Czerkinsky CC et al 1983 J Immunol Methods 65(1-2), 109-121), ELISA (Yalow, R. S. and S. A. Berson 1960, J. Clin Invest 39, 1157-1175), intracellular cytokine staining (Sander, Bet al 1991 Immunol Rev 119 65-93); an increase cellular division measured by thymidine incorporation (Johnson, H. A et al., 1960 Lab Invest 9 460-465), BrdU uptake (Russo, A et al 1984 Cancer Res 44(4), 1702-1705) or CFSE dilution (Lyons, A. B. and C. R. Parish 1994 J Immunol Methods 171(1), 131-137); or increased production of antibodies by ELISA or LIPs assay (Burbelo, P. D et al 2009 J Vis Exp, 32). Alternatively the ability of variant li sequences to improve the immune response could be determined in vitro by demonstrating increased antigen expression by immunohistochemistry (Ramos-Vara, J. A. 2005 Vet Pathol 42(4), 405-426).

In some embodiments, the antigen may be directly fused to the li molecule at the C-terminus of the li molecule. In other embodiments a flexible peptide linker is included between the antigen and the li molecule. A flexible peptide linker is a series of amino acids which connects two defined regions, in this instance the antigen and the li molecule, and allows the two defined regions to move. Preferably the linker allows the two regions to have locational freedom. Preferably the linker allows the regions it links to form their preferred configuration whilst still being linked. Any suitable flexible linker may be employed and a flexible linker may incorporate additional functionality. For example a flexible linker may also act as a tag for antibody recognition, and may be, e.g. a poly His tag.

Constructs of the invention may be any suitable nucleic acid construct capable of being used to immunise a human or non-human animal. In some embodiments the construct is a DNA plasmid. In some embodiments the construct is linear or single stranded DNA. In some embodiments the construct is RNA based. In some embodiments the construct is a viral vector. Viruses or bacteria that are non-replicating or replication impaired are preferred, and may have arisen naturally or may have been produced artificially, for example, by genetic manipulation.

Viral vectors according to the present invention may be made from a modified viral genome, i.e. the actual DNA or RNA forming the viral genome, and may be introduced in naked form, or in the form of a complete or partially complete virus particle.

The virus from which the viral vector is derived is selected from the non-exhaustive group of: adenoviruses such as chimpanzee adenoviruses, eg. ChAdOx1 or ChAd63, retroviruses, alpha viruses, yellow fever viruses, adeno-associated viruses, herpes viruses, vesicular stomatitis viruses, vaccinia viruses, and vaccinia derived viruses such as MVA or NYVAC, foamy viruses, rubella virus, VZV virus, cytomegaloviruses, Semliki forest virus, poxviruses, avipox viruses, such as canary pox or fowl pox, or influenza viruses. Adenoviral vectors may include non-replication or replication impaired human or simian adenoviruses. Such viral vectors are well known in the art.

Preferably if the vector is a viral vector it is an adenovirus or an MVA virus.

A bacterial vector may comprise recombinant Salmonella, recombinant Listeria, recombinant Shigella or recombinant BCG.

The invention also includes a cell comprising the nucleic acid construct as disclosed herein. Such a recombinant cell can be used as a tool for in vitro research, as a delivery vehicle for the nucleic acid construct or as part of a gene-therapy regime. The nucleic acid construct according to the invention can be introduced into cells by techniques well known in the art and which include microinjection of DNA into the nucleus of a cell, transfection, electroporation, lipofection/liposome fusion and particle bombardment. Suitable cells include autologous and non-autologous cells, and may include xenogenic cells.

In a preferred embodiment the nucleic acid construct of the present invention is comprised within an antigen presenting cell (APC). Any cell that presents antigens on its surface in association with an MHC molecule is considered an antigen presenting cell. Such cells include but are not limited to macrophages, dendritic cells, B cells, hybrid APCs, and foster APCs. Methods of making hybrid APCs are well known in the art.

In a more preferred embodiment the APC is a professional antigen presenting cell and most preferably the APC is an MHC-I and/or MHC-II expressing cell. The APC according to any of the above may be a stem cell obtained from a patient. After introducing the nucleic acid construct of the invention, the stem cell may be reintroduced into the patient in an attempt to treat the patient of a medical condition. Preferably, the cell isolated from the patient is a stem cell capable of differentiating into an antigen presenting cell.

The nucleic acid construct of the invention may be comprised within a delivery vehicle, for example polyethylenimine. Delivery vehicles are generally used for expression of the sequences encoded within the nucleic acid construct and/or for the intracellular delivery of the construct or the polypeptide encoded therein.

The nucleic acid construct may be transferred into cells in vivo or ex vivo; the latter by removing the target tissue (i.e., liver cells or white blood cells) from the patient, transferring the construct in vitro and then replanting the transduced cells into the patient.

Methods of non-viral delivery include physical (carrier-free delivery) and chemical approaches (synthetic vector-based delivery). Physical approaches, including needle injection, gene gun, jet injection, electroporation, ultrasound, and hydrodynamic delivery, employ a physical force that permeates the cell membrane and facilitates intracellular gene transfer. Said physical force may be electrical or mechanical.

The chemical approaches use synthetic or naturally occurring compounds as carriers to deliver the transgene into cells. The most frequently studied strategy for non-viral gene delivery is the formulation of DNA into condensed particles by using cationic lipids or cationic polymers. The DNA-containing particles are subsequently taken up by cells via endocytosis, macropinocytosis, or phagocytosis in the form of intracellular vesicles, from which a small fraction of the DNA is released into the cytoplasm and migrates into the nucleus, where transgene expression takes place.

It is within the scope of the present invention that the delivery vehicle is a vehicle selected from the group of: RNA based vehicles, DNA based vehicles; vectors, lipid based vehicles, polymer based vehicles and virally derived DNA or RNA vehicles.

Examples of chemical delivery vehicles include, but are not limited to: biodegradable polymer microspheres, lipid based formulations such as liposome carriers, cationically charged molecules such as liposomes, calcium salts or dendrimers, lipopolysaccharides, polypeptides and polysaccharides.

Alternative physical delivery methods may include aerosol instillation of a naked nucleic acid construct on mucosal surfaces, such as the nasal and lung mucosa; topical administration of the nucleic acid construct to the eye and mucosal tissues; and hydration such as stromal hydration by which saline solution is forced into the corneal stroma of the eye.

The constructs of the invention, or cells or other delivery vehicles containing the constructs of the invention may be used as vaccines, and may be used in therapy by immunisation. Preferably the resulting vaccines, cells and constructs may be used in the prevention or treatment of infectious diseases, or cancer.

The antigen encoded by the construct may be a protein or peptide or fragment of a protein or peptide. The antigen may include segments or epitopes from one or more protein and may include one or more segments or epitopes from the same protein. In effect, the antigen may be a multi-part antigen comprising antigens from more than one source. The antigen may be referred to as the at least one antigen' and this is intended to convey that such multi-part antigens are included. However, references to the antigen (in the singular) should not be taken as excluding multi-part antigens. Unless otherwise directed, all embodiments herein are suitable for use with single antigens or multi-part antigens.

Antigens useful in the invention may be derived from pathogenic organisms, cancer-specific polypeptides and antigens, and proteins or peptides associated with an abnormal physiological response.

In some embodiments the at least one antigen may be derived from any of the following types of pathogens: virus, micro-organisms and parasites. This includes pathogens of any animal known. In preferred embodiments, the antigen is derived from a human pathogen. In general, any antigen that is found to be associated with a human pathogen or disease may be used.

In other embodiments, the at least one antigen may be derived from an avian pathogen i.e. a pathogen that specifically targets birds or fowls. In preferred embodiments the antigen is derived from a pathogen of chicken (gallus gallus domesticus). In general, any antigen that is found to be associated with an avian pathogen may be used.

In yet other embodiments, the at least one antigen may be derived from a piscine pathogen i.e. a pathogen that specifically targets fish. Preferably, the antigen is derived from a pathogen of a fish that may be bred in captivity. In general, any antigen that is found to be associated with a piscine pathogen may be used.

In a some embodiments the at least one antigen may originate from, but is not limited to any of the following families of virus: Adenovirus, arenaviridae, astroviridae, bunyaviridae, caliciviridae, coronaviridae, flaviviridae, herpesviridae, orthomyxoviridae, paramyxoviridae, picornaviridae, poxviridae, reoviridae, retroviridae, rhabdoviridae and togaviridae. More specifically the antigen may be derived from any of the following virus: Influenza A such as H1 N1, H1 N2, H3N2 and H5N1 (bird flu), Influenza B, Influenza C virus, Hepatitis A virus, Hepatitis B virus, Hepatitis C virus, Hepatitis D virus, Hepatitis E virus, Rotavirus, any virus of the Norwalk virus group, enteric adenoviruses, parvovirus. Dengue fever virus, Monkey pox, Mononegavirales. Lyssavirus such as rabies virus. Lagos bat virus, Mokola virus, Duvenhage virus, European bat virus 1 & 2 and Australian bat virus, Ephemerovirus, Vesiculovirus, Vesicular Stomatitis Virus (VSV), Herpesviruses such as Herpes simplex virus types 1 and 2, varicella zoster, cytomegalovirus, Epstein-Bar virus (EBV), human herpesvirusses (HHV), human herpesvirus type 6 and 8. Human immunodeficiency virus (HIV), papilloma virus, murine gammaherpesvirus, Arenaviruses such as Argentine hemorrhagic fever virus, Bolivian hemorrhagic fever virus. Sabia-associated hemorrhagic fever virus. Venezuelan hemorrhagic fever virus, Lassa fever virus. Machupo virus, Lymphocytic choriomeningitis virus (LCMV), Bunyaviridiae such as Crimean-Congo hemorrhagic fever virus. Hantavirus, hemorrhagic fever with renal syndrome causing virus, Rift Valley fever virus, Filoviridae (filovirus) including Ebola hemorrhagic fever and Marburg hemorrhagic fever, Flaviviridae including Kaysanur Forest disease virus, Omsk hemorrhagic fever virus, Tick-borne encephalitis causing virus and Paramyxoviridae such as Hendra virus and Nipah virus, variola major and variola minor (smallpox), alphaviruses such as Venezuelan equine encephalitis virus, eastern equine encephalitis virus, western equine encephalitis virus, SARS-associated coronavirus (SARS-CoV), West Nile virus, any encephaliltis causing virus.

In some embodiments the at least one antigen is derived from a virus selected from the group of: HIV, Hepatitis C virus, influenza virus, herpes virus, Lassa. Ebola, smallpox, Bird flu, filovirus, Marburg, and papilloma virus.

In other embodiments the at least one antigen is selected from the group of and/or may be at least one antigenic fragment of any of the following: vesicular stomatitis virus glycoprotein (VSV-GP): Influenza A NS-1 (non-structural protein 1), M1 (matrix protein 1), NP (nucleoprotein), NEP_{;} M2, M2e, HA, NA, PA, PB1 PB2, PB1 -F2; LCMV NP, LCMV GP; Ebola GP, Ebola NP; HIV antigens tat, vif, rev, vpr, gag, poi, nef, env, vpu; Sly antigens tat, vif, rev, vpr, gag, pol, nef, env; murine gammaherpesvirus M2, M3 and ORF73 (such as MHV-68 M2, M3 and ORF73); chicken Ovalbumin (OVA); or a helper T-cell epitope. It is within the scope of the invention to combine two or more of any of the herein mentioned antigens.

Other embodiments include at least one antigen from a micro-organism. More specifically the at least one antigen may be derived from the one of the following from a non-exhaustive list: Anthrax (Bacillus anthracis), Mycobacterium tuberculosis, Salmonella (Salmonella gallinarum, S. pullorum, S. typhi, S. enteridtidis, S. paratyphi, S. dublin, S. typhimurium), Clostridium botulinum, Clostridium perfringens, Corynebacterium diphtheriae, Bordetella pertussis, Campylobacter such as Campylobacter jejuni, Crytococcus neoformans, Yersinia pestis, Yersinia enterocolitica, Yersinia pseudotuberculosis, Listeria monocytogenes, Leptospira species, Legionella pneumophila, Borrelia burgdorferi, Streptococcus species such as Streptococcus pneumoniae, Neisseria meningitides, Haemophilus influenzae, Vibrio species such as Vibrio cholerae O1, V. cholerae non-O1, V. parahaemolyticus, V. parahaemolyticus, V. alginolyticus, V. furnissii, V. carchariae, V. hollisae, V. cincinnatiensis, V. metschnikovii, V. damsela, V. mimicus, V. fluvialis, V. vulnificus, Bacillus cereus, Aeromonas hydrophila, Aeromonas caviae, Aeromonas sobria & Aeromonas veronii, Plesiomonas shigelloides, Shigella species such as Shigella sonnei, S. boydii, S. flexneri, and S. dysenteriae, Enterovirulent Escherichia coli EEC (Escherichia coli enterotoxigenic (ETEC), Escherichia coli enteropathogenic (EPEC), Escherichia coli O157:1-17 enterohemorrhagic (EHEC), Escherichia coli—enteroinvasive (EIEC)), Staphylococcus species, such as S. aureus and especially the vancomycin intermediate/resistant species (VISA/VRSA) or the multidrug resistant species (MRSA), Shigella species, such as S. flexneri, S. sonnei, S. dysenteriae, Cryptosporidium parvum, Brucella species such as B. abortus, B. melitensis, Bovis, B. suis, and B. canis, Burkholderia mallei and Burkholderia pseudomallei, Chlamydia psittaci, Coxiella burnetii, Francisella tularensis; Rickettsia prowazekii, Histoplasma capsulatum, Coccidioides immitis.

In some embodiments, the at least one antigen is from a micro-organism selected from the group of: Mycobacterium tuberculosis, Bacillus anthracis, Staphylococcus species; and Vibrio species.

In some embodiments the invention relates to a nucleic acid construct, wherein the at least one antigenic protein or peptide encoded is from a parasite.

In some embodiments the invention relates to a nucleic acid construct comprising combinations of at least two antigenic proteins or peptides from any of the abovementioned pathogens.

Preferably the antigen is derived from, but not limited to, a parasite selected from the group of: Plasmodium species such as Plasmodium malariae, Plasmodium ovale; Plasmodium vivax, Plasmodium falciparum, Endolimax nana, Giardia lamblia, Entamoeba histolytica; Cryptosporidum parvum, Blastocystis hominis, Trichomonas vaginalis, Toxoplasma gondii, Cyclospora cayetanensis, Cryptosporidium muris, Pneumocystis carinii, Leishmania donovani, Leishmania tropica, Leishmania braziliensis, Leishmania mexicana, Acanthamoeba species such as Acanthamoeba castellanii, and A. culbertsoni, Naegleria fowleri, Trypanosoma cruzi, Trypanosoma brucei rhodesiense, Trypanosoma brucei gambiense, Isospora belli, Balantidium coli, Roundworm (Ascaris lumbricoides), Hookworm (Necator Americanus, Ancylostoma duodenal), Pinworm (Enterobius vermicularis), Roundworm (Toxocara canis, Toxocara cati), Heart worm (Dirofilaria immitis), Strongyloides (Stronglyoides stercoralis), Trichinella (Trichinella spiralis), Filaria (Wuchereria bancrofti, Brugia malayi, Onchocerca volvulus, Loa boa, Mansonella streptocerca, Mansonella perstans; Mansonella ozzardij, and Anisakine larvae (Anisakis simplex (herring worm), Pseudoterranova (Phocanema, Terranova) decipiens (cod or seal worm), Contracaecum species, and Hysterothylacium (Thynnascaris species) Trichuris trichiura, Beef tapeworm (Taenia saginata), Pork tapeworm (Taenia solium), Fish tapeworm (Diphyllobothrium latum), and Dog tapeworm (Dipylidium caninum), Intestinal fluke (Fasciolopsis buski), Blood fluke (Schistosoma japonicum, Schistosoma mansoni) Schistosoma haematobium), Liver fluke (Clonorchis sinensis), Oriental lung fluke (Paragonimus westermani), and Sheep liver fluke (Fasciola hepatica), Nanophyetus salmincola and N. schikhobalowi.

In a preferred embodiment of the invention the at least one antigenic protein or peptide is from a parasite selected from the group of: Plasmodium species, Leishmania species, and Trypanosoma species.

In a further aspect of the present invention the at least one antigen is derived from diseases or agents that infect domestic animals, especially commercially relevant animals such as pigs, cows, horses, sheep, goats, llamas, rabbits, mink, mice, rats, dogs, cats, ferrets, poultry such as chicken, turkeys, pheasants and others, fish such as trout, salmon, cod and other farmed species. Examples of diseases or agents here of from which at least one antigen or antigenic sequence may be derived include, but are not limited to: Multiple species diseases such as: Anthrax, Aujeszky's disease, Bluetongue, Brucellosis such as: Brucella abortus, Brucella melitensis or Brucella suis; Crimean Congo haemorrhagic fever, Echinococcosislhydatidosis, virus of the family Picornaviridae, genus Aphthovirus causing Foot and Mouth disease especially any of the seven immunologically distinct serotypes: A, O, C. SAT1, SAT2, SAT3, Asiai , or Heartwater, Japanese encephalitis, Leptospirosis, New world screwworm (Cochliomyia hominivorax), Old world screwworm (Chrysomya bezziana), Paratuberculosis. Q fever, Rabies, Rift Valley fever, Rinderpest, Trichinellosis, Tularemia, Vesicular stomatitis or West Nile fever; Cattle diseases such as: Bovine anaplasmosis, Bovine babesiosis, Bovine genital campylobacteriosis, Bovine spongiform encephalopathy, Bovine tuberculosis, Bovine viral diarrhoea. Contagious bovine pleuropneumonia, Enzootic bovine leukosis, Haemorrhagic septicaemia, Infectious bovine rhinotracheitis/infectious pustular vulvovaginitis, Lumpy skin disease, Malignant catarrhal fever, Theileriosis, Trichomonosis or Trypanosomosis (tsetse-transmitted); Sheep and goat diseases such as: Caprine arthritis/encephalitis, Contagious agalactia, Contagious caprine pleuropneumonia, Enzootic abortion of ewes (ovine chlamydiosis), Maedi-visna, Nairobi sheep disease, Ovine epididymitis (Brucella ovis), Peste des petits ruminants, Salmonellosis (S. abortusovis), Scrapie, Sheep pox and goat pox; Equine diseases such as: African horse sickness, Contagious equine metritis, Dourine, Equine encephalomyelitis (Eastern), Equine encephalomyelitis (Western), Equine infectious anaemia, Equine influenza, Equine piroplasmosis, Equine rhinopneumonitis, Equine viral arteritis, Glanders, Surra (Trypanosoma evansi) or Venezuelan equine encephalomyelitis; Swine diseases such as: African swine fever, Classical swine fever, Nipah virus encephalitis, Porcine cysticercosis, Porcine reproductive and respiratory syndrome, Swine vesicular disease or Transmissible gastroenteritis; Avian diseases such as: Avian chlamydiosis, Avian infectious bronchitis, Avian infectious laryngotracheitis, Avian mycoplasmosis (M. gallisepticum), Avian mycoplasmosis (M. synoviae), Duck virus hepatitis, Fowl cholera, Fowl typhoid, Highly pathogenic avian influenza this being any lnfluenzavirus A or B and especially H5N1 Infectious bursal disease (Gumboro disease), Marek's disease, Newcastle disease, Pullorum disease or Turkey rhinotracheitis; Lagomorph and rodent diseases such as: Virus enteritis, Myxomatosis or Rabbit haemorrhagic disease; Fish diseases such as: Epizootic haematopoietic necrosis, Infectious haematopoietic necrosis, Spring viraemia of carp, Viral haemorrhagic septicaemia, Infectious pancreatic necrosis, Infectious salmon anaemia, Epizootic ulcerative syndrome, Bacterial kidney disease (Renibacterium salmoninarum), Gyrodactylosis (Gyrodactylus salaris), Red sea bream iridoviral disease; or other diseases such as Camelpox or Leishmaniosis.

In a preferred embodiment of the invention the at least one antigenic protein or peptide is from Aujeszky's disease, Foot and mouth disease, Vesicular stomatitis virus, Avian influenza or Newcastle disease.

Yet a preferred embodiment of the present invention relates to the at least one antigenic protein or peptide or fragment of said antigenic protein or peptide being an antigenic peptide or protein with at least 85% identity to any of the above described antigens. The homology or identity between amino acids may be calculated by any of the previously mentioned BLOSUM scoring matrices.

Many proteinlglycoproteins have been identified and linked to certain types of cancer; these are referred to as cancer-specific polypeptides, tumor-associated antigens or cancer antigens. In general, any antigen that is found to be associated with cancer tumors may be used. One way in which cancer-specific antigens may be found is by subtraction analyses such as various microarray analyses, such as DNA microarray analysis. The gene-expression pattern (as seen in the level of RNA or protein encoded by said genes) between healthy and cancerous patients, between groups of cancerous patients or between healthy and cancerous tissue in the same patient is compared. The genes that have approximately equal expression levels are “subtracted” from each other leaving the genes/gene products that differ between the healthy and cancerous tissue. This approach is known in the art and may be used as a method of identifying novel cancer antigens or to create a gene-expression profile specific for a given patient or group of patients. Antigens thus identified, both single antigen and the combinations in which they may have been found fall within the scope of the present invention. Preferably the at least one antigen of the present invention is derived from, but not limited to, a cancer-specific polypeptide selected from the group of: MAGE-3, MAGE-1, gpl 00, gp75, TRP-2, tyrosinase, MART-1, CEA, Ras, p53, B-Catenin, gp43, GAGE-1, BAGE-1, PSA, PSMA, PSCA, STEAP, PAP, MUC-1 2, 3, and HSP-70, TRP-1, 5T4, gp100/pme117, beta-HCG. Ras mutants, p53 mutants, HMW melanoma antigen, MUC-18, HOJ-1, cyclin-dependent kinase 4 (Cdk4), Caspase 8, HER-2/neu, Bcr-Abl tyrosine kinase, carcinoembryonic antigen (CEA), telomerase, SV40 Large T. Human papilloma virus HPV type 6, 1 1 , 16, 18, 31 and 33; HPV derived viral oncogene E5, E6, E7 and L1; Survivin, Bcl-XL, MCL-1 and Rho-C.

In a preferred embodiment of the invention, the at least one antigenic protein or peptide or fragment of an antigenic protein or peptide is from a cancer-specific polypeptide selected from the group of: HPV derived viral oncogene E5 E6, E7 and L1; Survivin, Bcl-XL, MCL-1 and Rho-C.

A further embodiment of the invention relates to a nucleic acid construct, wherein the at least one antigenic protein or peptide or fragment of an antigenic protein or peptide is from a polypeptide associated with an abnormal physiological response. Such an abnormal physiological response includes, but is not limited to autoimmune diseases, allergic reactions, cancers and congenital diseases. A non-exhaustive list of examples hereof includes diseases such rheumatoid arthritis, systemic lupus erythematosus, multiple sclerosis, psoriasis and Crohn's disease.

Preferably the immunogenic or vaccine composition is for use in therapeutic or prophylactic treatments or both.

The immune response elicited by any method of the invention may be therapeutic or prophylactic or both.

An immunogenic or vaccine composition according to the invention may be for oral, systemic, parenteral, topical, mucosal, intramuscular, intraperitoneal, intradermal, subcutaneous, intranasal, intravaginal, sublingual, or inhalation administration.

A composition according to the invention may be administered to a subject/organism in the form of a pharmaceutical composition. In addition to the immunogenic or vaccine composition, a pharmaceutical composition preferably comprises one or more physiologically and/or pharmaceutically effective carriers, diluents, excipients or auxiliaries which facilitate processing and/or delivery of the antigen and/or adjuvant.

Determination of an effective amount of an immunogenic or vaccine composition for administration to an organism is well within the capabilities of those skilled in the art. For example, for mouse to humans, a DNA vaccination dose may comprise from about 0.1 μg to about 10 mg, For an adenoviral vector the vaccination dose may be between about 1×10⁶ and 1×10¹⁶ viral particles per animal. For an MVA vector the vaccination dose may be between about 1×10² and 1×10¹⁰ pfu per animal,

A composition according to the invention may be used in isolation, or it may be combined with one or more other immunogenic or vaccine compositions, and/or with one or more other therapeutic regimes.

According to a further aspect the invention provides a kit for use in inducing an immune response in an organism, comprising an immunogenic or vaccine composition according to the invention and instructions relating to administration.

According to a yet further aspect, the invention provides a pharmaceutical composition comprising an immunogenic or a vaccine composition according to the invention and one or more physiologically effective carriers, diluents, excipients or auxiliaries.

According to another aspect, the invention provides the use of an immunogenic composition according to the invention in the preparation of a medicament for the treatment and/or prevention of infection and/or disease related to the antigen encoded by the vector in the immunogenic composition.

Where the antigen encoded by the vector in the composition is from a pathogen, the medicament may be intended/used to confer protection from infection and/or from disease caused by the pathogen from which the antigen of interest is derived. Alternatively, where the antigen encoded by the vector in the composition is a cancer antigen or an antigen associated with a particular disease, the medicament may be intended/used to confer protection from, and/or to treat, the cancer or the particular disease from which the antigen is derived.

According to another aspect the invention provides the use of an immunogenic composition according to the invention in the treatment and/or prevention of infection or disease related to the antigen encoded by the vector in the immunogenic composition.

Preferably, in a use according to the invention the composition or medicament induces an immune response when administered to an organism.

Preferably the organism is a human or non-human mammal or a bird such as a chicken. A non-human mammal may include a horse, cow, sheep, pig, goat, mouse, rat, monkey or chimpanzee.

In addition to their potential use as vaccines, immunogenic compositions according to the invention may be useful a) as diagnostic reagents: b) in adoptive T cell therapy protocols: and c) as a measure of immune competence of the vaccine.

The immune response induced in an organism may be a cellular immune response and/or a humoral immune response. If a cellular immune response is induced, the composition may, when administered to an organism, induce a T cell response against an antigen encoded by the vector in the composition. Preferably the T cell response is a CD8+ and/or a CD4+ T cell response. Preferably the immune response is protective, that is, it serves to protect, either reduce or prevent, the organism from developing an infection or disease related to the antigen encoded by the vector in the composition.

The immune response may be assessed by determining antigen-specific IFNγ secretion levels by lymphocytes, or by assaying for other cytokines secreted/induced in an antigen-specific manner. Other cytokines which may be secreted/induced in an antigen-specific manner include IL-2. IL-4, IL-12, and TNF-alpha. The aforementioned methods are just some examples of how induction of the cellular immune system may be monitored, and are not intended to be exhaustive.

The terms “non-replicating” or “replication impaired” as used herein mean that the viral vector is not capable of replication to any significant extent in a host organism, and in particular is unable to cause serious infection in the host. The host organism is preferably a human, wherein the terms “non-replicating” or “replication impaired” mean that the vector is not capable of replication to any significant extent in normal human cells.

Replication of a virus, and thus a viral vector, can be measured in two ways: (i) DNA synthesis, and (ii) viral titre. For adenovirus a non-replicating or a replication impaired viral vector may exhibit a significant reduction in viral titre on infection of cells, such as HeLa cells, which are not permissive for the replication of the replication-deficient adenovirus. For poxvirus a non-replicating or a replication impaired viral vector may exhibit a 2 log reduction in viral titre in HELA cells (a human cell line) compared to the Copenhagen strain of the vaccinia virus. Examples of poxviruses which fall within this definition are MVA, NYVAC and avipox, while a virus which falls outside this definition is the attenuated vaccinia strain M7.

Preferably the viral vector is based on a virus selected from the group comprising adenoviruses; vaccinia derived viruses, such as, MVA or NYVAC; avipox viruses, such as, canary pox or fowl pox; alpha viruses; herpes viruses; flaviviruses; retroviruses and influenza viruses, Adenoviral vectors may include non-replication or replication impaired human or simian adenoviruses.

Preferably the viral vector is an adenovirus such as the chimpanzee adenovirus ChAdOx1 or an orthopox virus such as the MVA or an avipoxvirus vector. Preferably the viral vector is not the fowl pox virus.

Viruses that are non-replicating or replication impaired may have arisen naturally or they may have been produced artificially, for example, by genetic manipulation.

The antigen may be naturally expressed by the vector. For example, if the vector is an adenovirus, the antigen may be an adenovirus protein which may confer immunity against subsequent infection and/or disease caused by an adenovirus of the same or similar strain. Alternatively, or additionally, the antigen may be exogenous to the vector.

The vector may encode one or more antigens. If the vector encodes more than one antigen, the antigens may be derived from the same pathogen or disease, or from different pathogens or diseases.

The skilled person will understand that optional features of one embodiment or aspect of the invention may be applicable, where appropriate, to other embodiments or aspects of the invention.

The invention will now be described further in the following non-limiting examples, with reference to the figures:

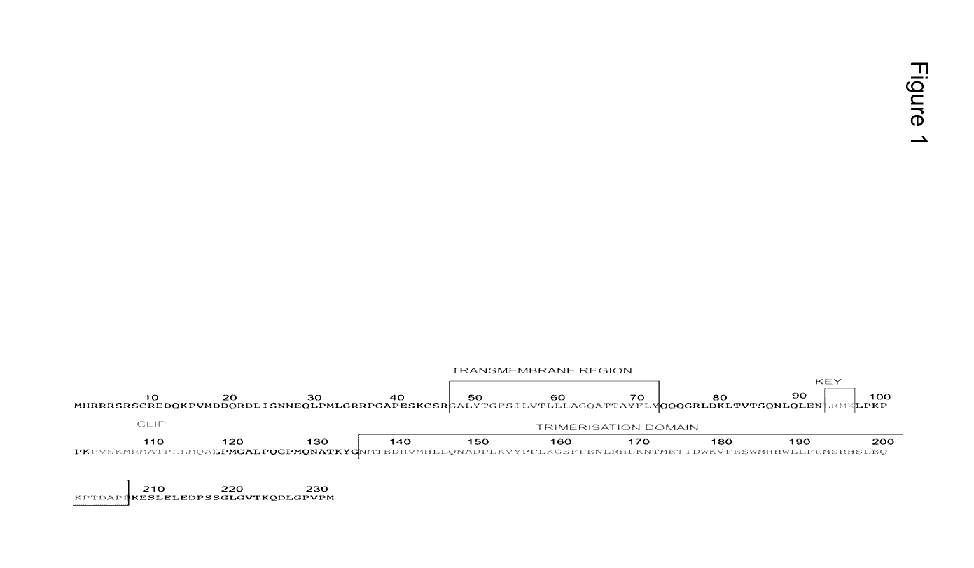

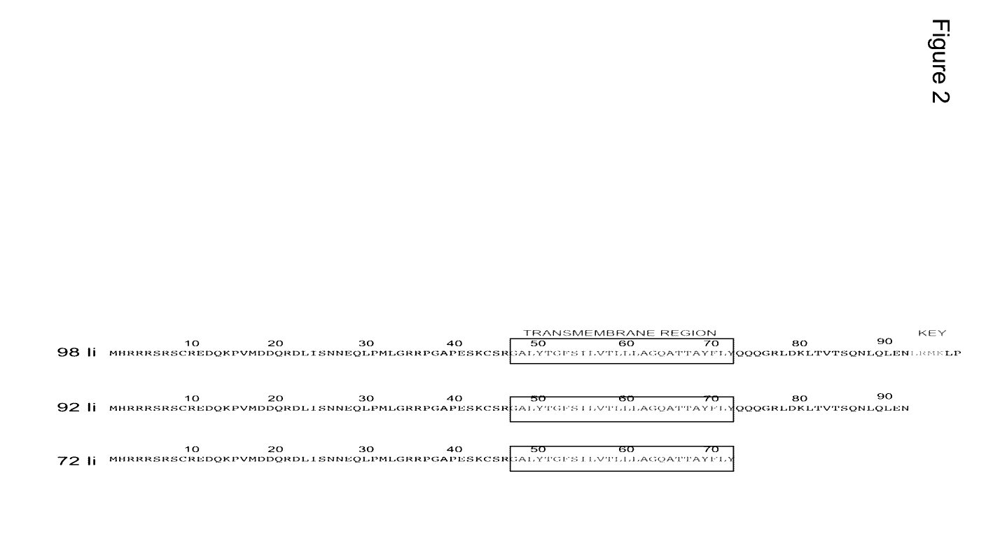

FIG. 1 shows the human Ii sequence SEQ ID NO: 13 (NCBI RefSeq NP 004346.1; isoform b) denoting the transmembrane domain, KEY, CLIP and trimerisation regions. SEQ ID NO.1 is equivalent to residues 1 to 98 of the sequence shown in FIG. 1.

FIG. 2 shows three C-terminally truncated human li sequences. 98 li (SEQ ID NO: 1) is SEQ ID NO.1. 92 li (SEQ ID NO 15) is amino acids 1 to 92 of SEQ ID NO.1 and 72 li (SEQ ID NO: 16) is amino acids 1 to 72 of SEQ ID NO.1.

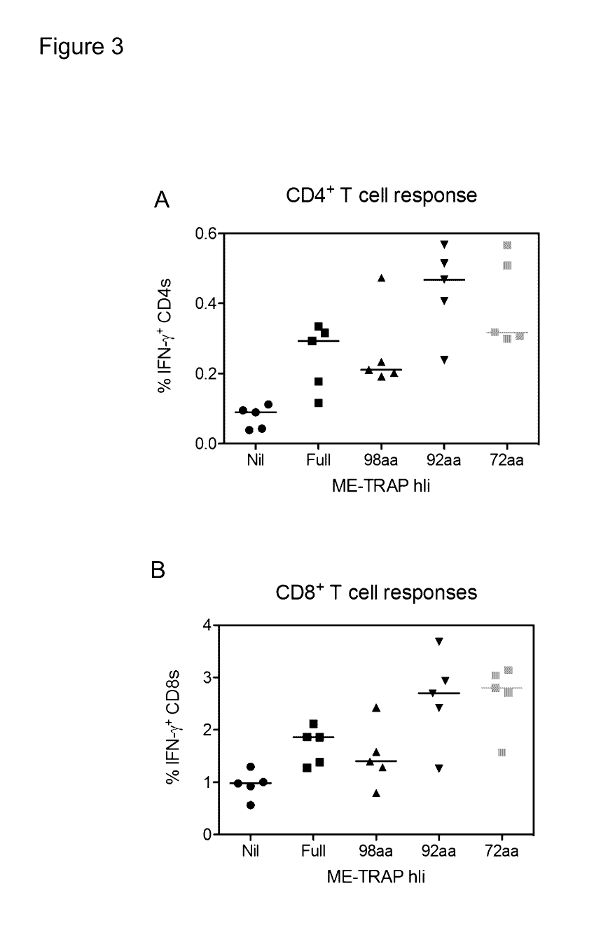

FIG. 3 shows Balb/c mice vaccinated with 10⁷ infectious units of ChAd.hli-ME-TRAP. 2 weeks later spleens were harvested and after 6 hours of re-stimulation with TRAP peptides, production of IFN-γ was measured by intracellular cytokine staining. Each mouse is represented by a single point, with lines showing the median response per group. ‘Nil’ refers to mice vaccinated with the control virus expressing only ME-TRAP. ‘Full’, ‘98aa’, ‘92aa’ and ‘72aa’ refer to mice vaccinated with virus vector in which the hli (human li) fused to ME-TRAP is full length, 98 li, 92 li or 72 li from FIG. 2 respectively. A) CD4+ response; B) CD8 ⁺ response.

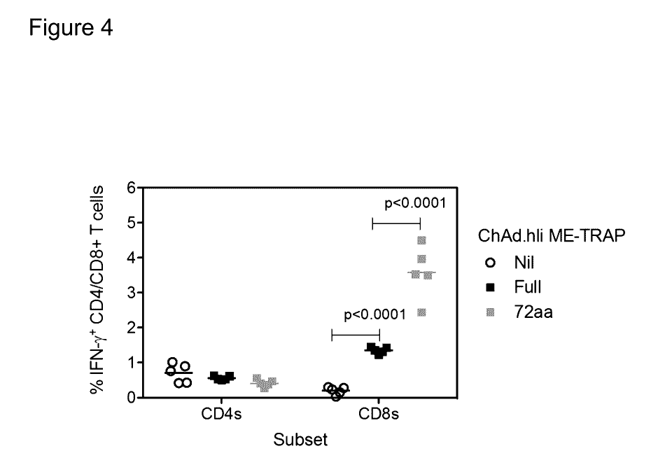

FIG. 4 shows C57BL/6J mice vaccinated with 10⁷ infectious units of ChAd.ME-TRAP (open circle), ChAd,hli-ME-TRAP (black squares) or ChAd.72aahli-ME-TRAP (grey squares). 2 weeks later spleens were harvested and after 6 hours of restimulation with TRAP peptides, production of IFN-γ was measured by intracellular cytokine staining. Each mouse is represented by a single point, with lines showing the median response per group. Data was analyzed with a two-way analysis of variance with a post-hoc Bonferroni test, p values indicate levels of significance. Designations of ‘Full’ and ‘72aa’ are as described above.

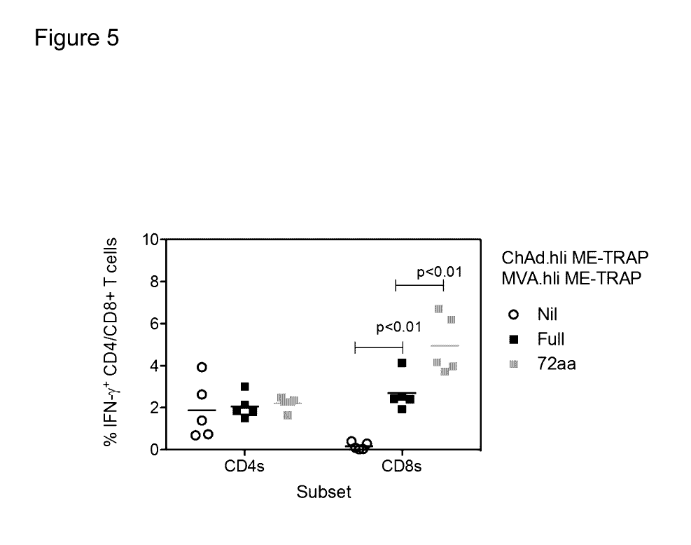

FIG. 5 shows C57BL/6J mice vaccinated with 10⁶ infectious units of ChAd.ME-TRAP (open circles), ChAd.hli-ME-TRAP (black squares) or ChAd.72aahli-ME-TRAP (grey squares) and 8 weeks later boosted with 10⁶ PFU MVA.ME-TRAP, MVA.hli-ME-TRAP or MVA.72aahli-ME-TRAP.1 week later spleens were harvested and after 6 hours of re-stimulation with TRAP peptides, production of IFN-γ was measured by intracellular cytokine staining. Each mouse is represented by a single point, with lines showing the median response per group. Data was analyzed with a two-way analysis of variance with a post-hoc Bonferroni test, p values indicate levels of significance. Designations of ‘Nil’, ‘Full’ and ‘72aa’ are as described above.

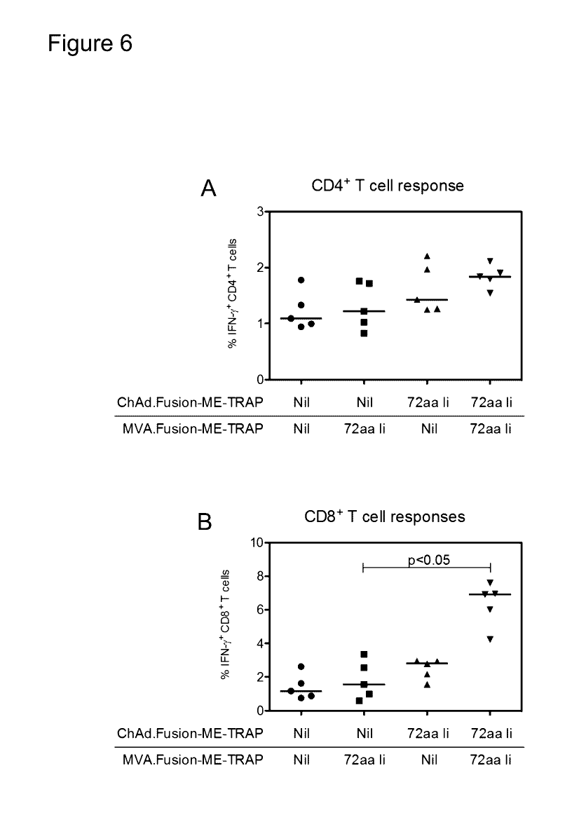

FIG. 6 shows C57BL/6J mice vaccinated with 10⁶ infectious units of ChAd.ME-TRAP and 8 weeks later boosted with 10⁶ PFU MVA. ME-TRAP. 1 week later spleens were harvested and after 6 hours of restimulation with TRAP peptides, production of IFN-γ was measured by intracellular cytokine staining. Each mouse is represented by a single point, with lines showing the median response per group. Data in each graph was analyzed with a one-way analysis of variance with a post-hoc Dunns multiple comparison test, p values indicate levels of significance. Designations of ‘Nil’ and ‘72aa’ are as described above.

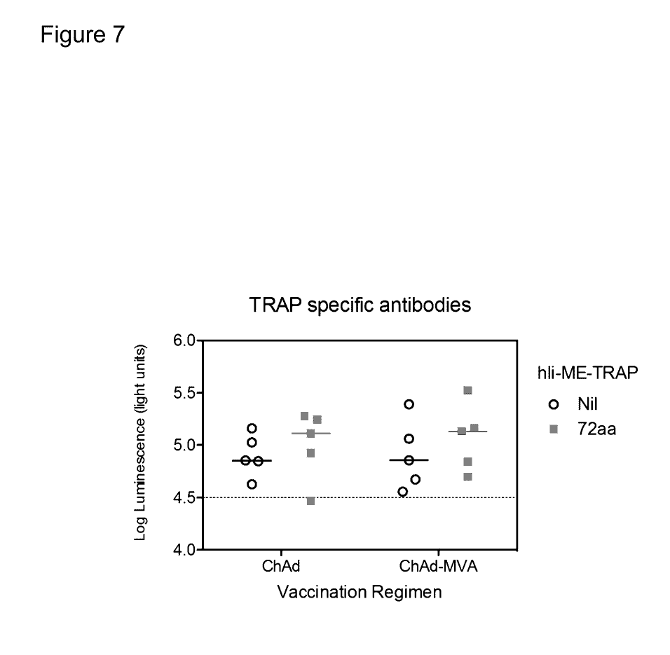

FIG. 7 shows C57BL/6J mice vaccinated with 10⁸ infectious units of ChAd.ME-TRAP and boosted 8 weeks later with 10⁶ PFU MVA.ME-TRAP. Serum was taken 3 weeks after ChAd vaccination and 1 week after MVA boost to measure TRAP specific antibodies by LIPs assay. Each mouse is represented by a single point, with lines showing the median response per group. The dotted line represents the background level as measured on serum from unvaccinated C57BL/6J mice.

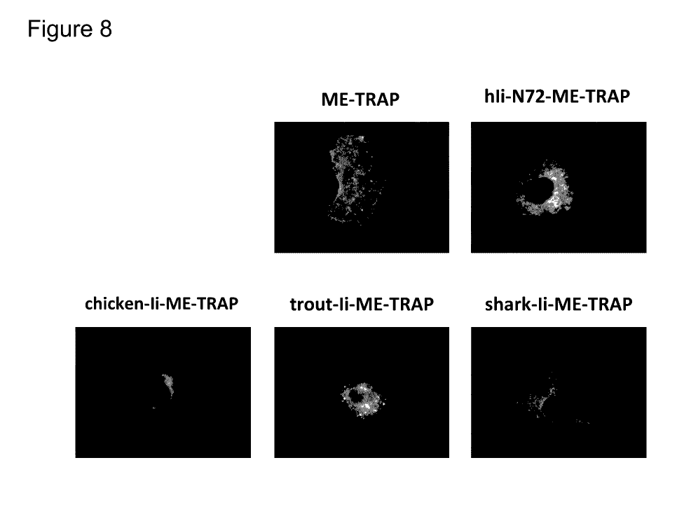

FIG. 8 shows A549 cells transfected with DNA plasmids encoding ME-TRAP, or ME-TRAP fused to truncated sequences of human, chicken, trout or shark li chain. 24 hours later, cells were stained for expression of surface and intracellular TRAP (green) expression. Fusion to the invariant chain is shown to alter TRAP expression to localised expression to cytoplamsic locations.

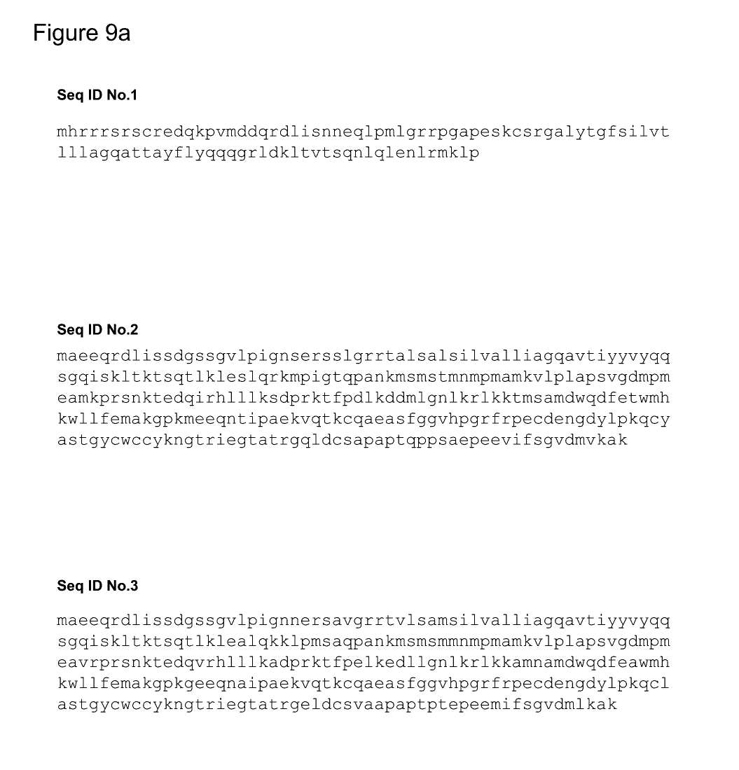

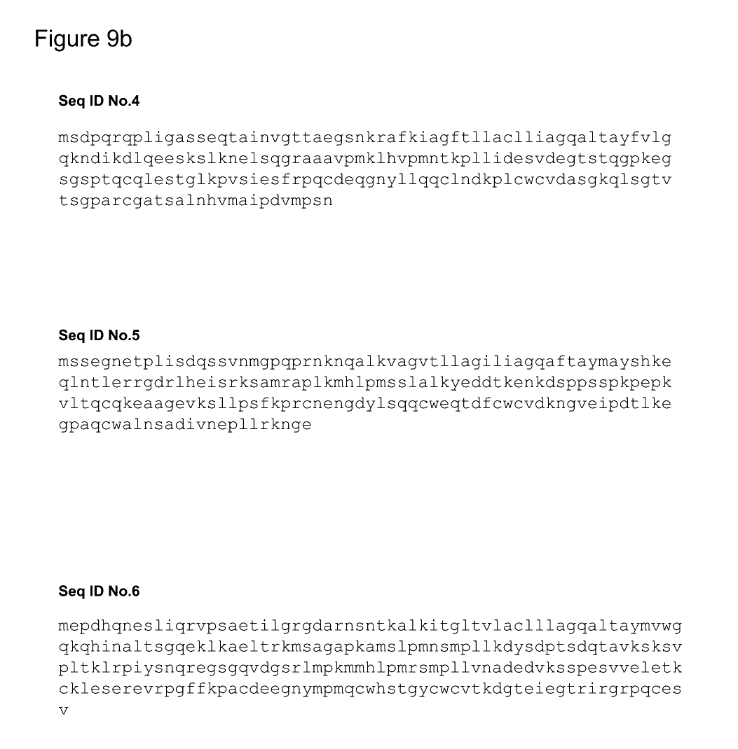

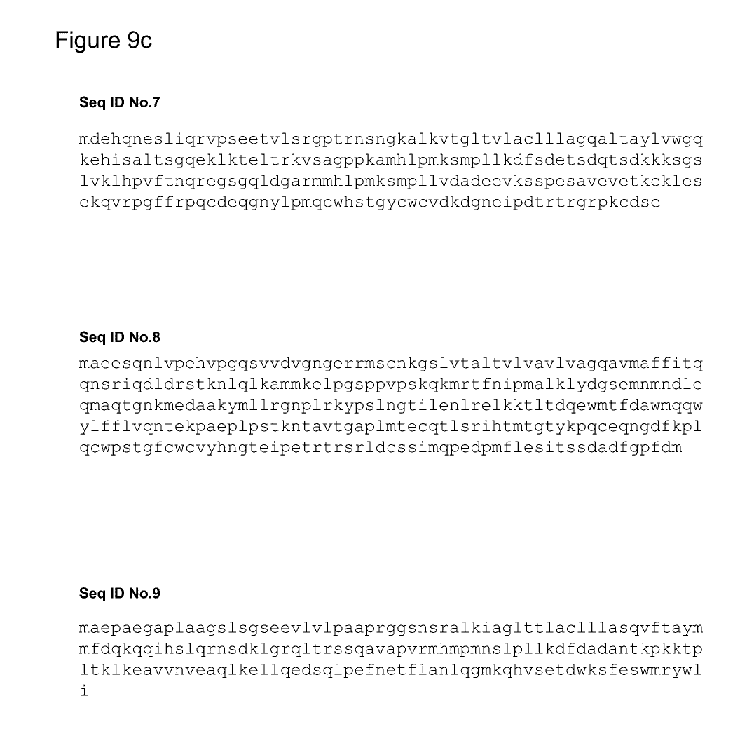

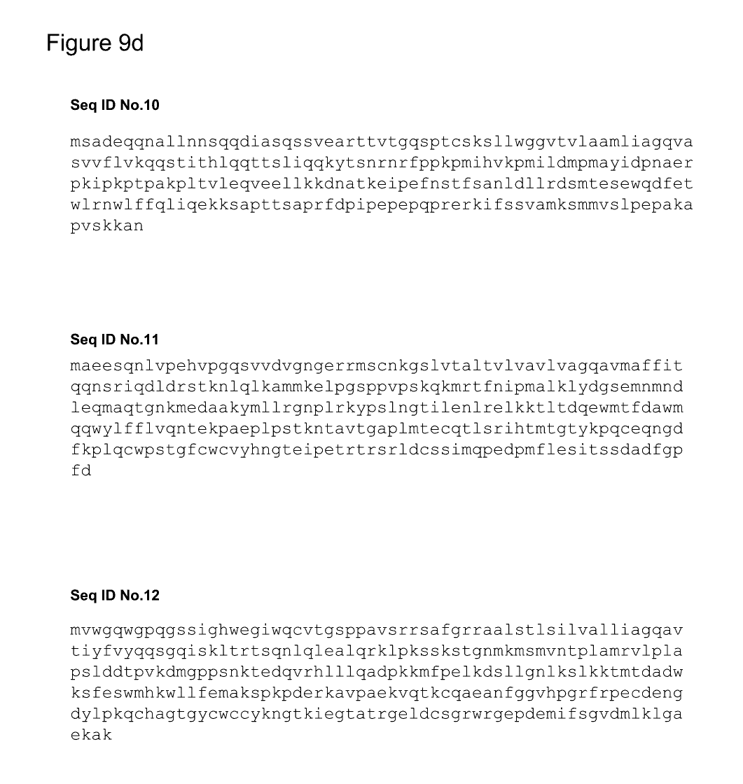

FIG. 9 shows the sequences of SEQ ID No.s 1 to 12. FIG. 9a shows SEQ ID No.1-3; FIG. 9b shows SEQ ID NO.4-6; FIG. 9c shows SEQ ID No. 7-9; FIG. 9d shows SEQ ID NO. 10-12

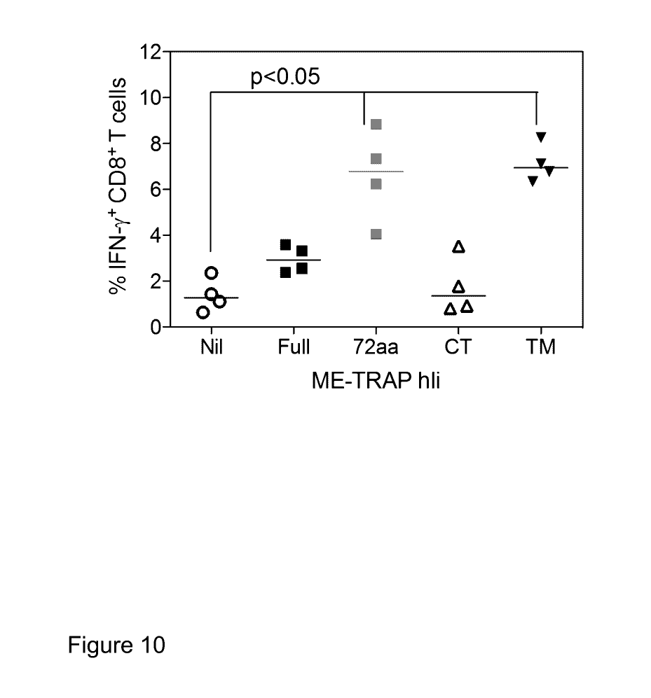

FIG. 10 shows the results of a study in which C57BL/6J mice were vaccinated with 10⁷ infectious units of ChAd.ME-TRAP (open circle) or ME-TRAP fused to the full length hli (closed square), truncated 72aa hli (SEQ ID NO: 16) (grey square), cytoplasmic only region of hli (open triangle) or transmembrane only region of hli (SEQ ID NO: 14, GALYTGFSILVTLLLAGQATTAYFLY, amino acids 47-72 of SEQ ID NO: 1) (closed triangle). 2 weeks later spleens were harvested and after 6 hours of re-stimulation with TRAP peptides, production of IFN-γ was measured by intracellular cytokine staining. Each mouse is represented by a single point, with lines showing the median response per group. Data was analyzed with a one-way analysis of variance with a post-hoc Kruskal-Wallis test, p values indicate levels of significance.

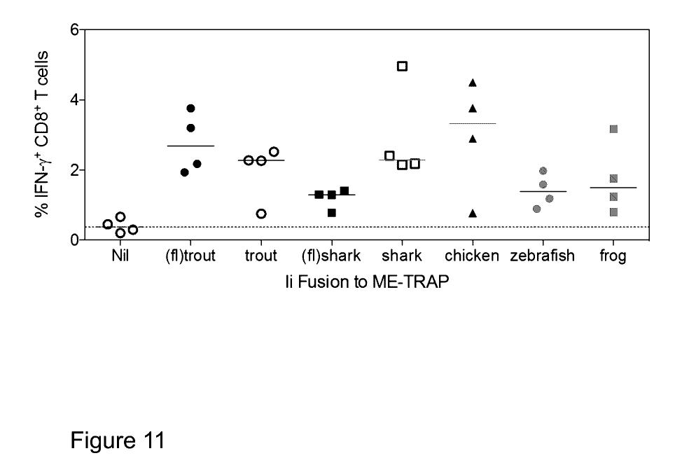

FIG. 11 shows the results of a study in which C57BLi6J mice were vaccinated with 10′ infectious units of ChAd.ME-TRAP (open circle) or ME-TRAP fused to full length trout (closed circle) or truncated trout (open circles), full length shark (closed square) or truncated shark (open square) , chicken (closed triangle), zebrafish (grey circle) or frog (grey square) li chain. 2 weeks later spleens were harvested and after 6 hours of re-stimulation with TRAP peptides, production of IFN-γ was measured by intracellular cytokine staining. Each mouse is represented by a single point, with lines showing the median response per group. Data was analyzed with a one-way analysis of variance with a post-hoc Kruskal-Wallis test, p values indicate levels of significance. The dotted line indicates the median response from ChAd.ME-TRAP vaccinated mice, “(fl)trout” is Seq ID No.4 shown in FIG. 9b “trout” is a truncated version of “(fl)trout”:

(SEQ ID NO 17)
MSDPQRQPLIGASSEQTAINVGTTAEGSNKRAFKIAGFTLLACLLIAG
QALTAYFVL,

(fl)shark” is Seq ID No.10 in FIG. 9d “shark” is a truncated version of “(fl)shark”:

(SEQ ID NO 18)
MSADEQQNALLNNSQQDIASQSSVEARTTVTGQSPTCSKSLLWGGV
TVLAAMLIAGQVASVVFLV,

“chicken” is a truncated version of Seq ID No.2 in FIG. 9a:

(SEQ ID NO: 19)
MAEEQRDLISSDGSSGVLPIGNSERSSLGRRTALSALSILVALLIAGQ
AVTIYYVY,

“zebrafish” is a truncated version of Seq ID No.5 in FIG. 9b:

(SEQ ID NO: 20)
MSSEGNETPLISDQSSVNMGPQPRNKNQALKVAGVTLLAGILIAGQA
FTAYMAY,

“frog” is a truncated version of Seq ID No.8 in FIG. 9c:

(SEQ ID NO: 21)
MAEESQNLVPEHVPGQSVVDVGNGERRMSCNKGSLVTALTVLVAVL
VAGQAVMAFFIT.

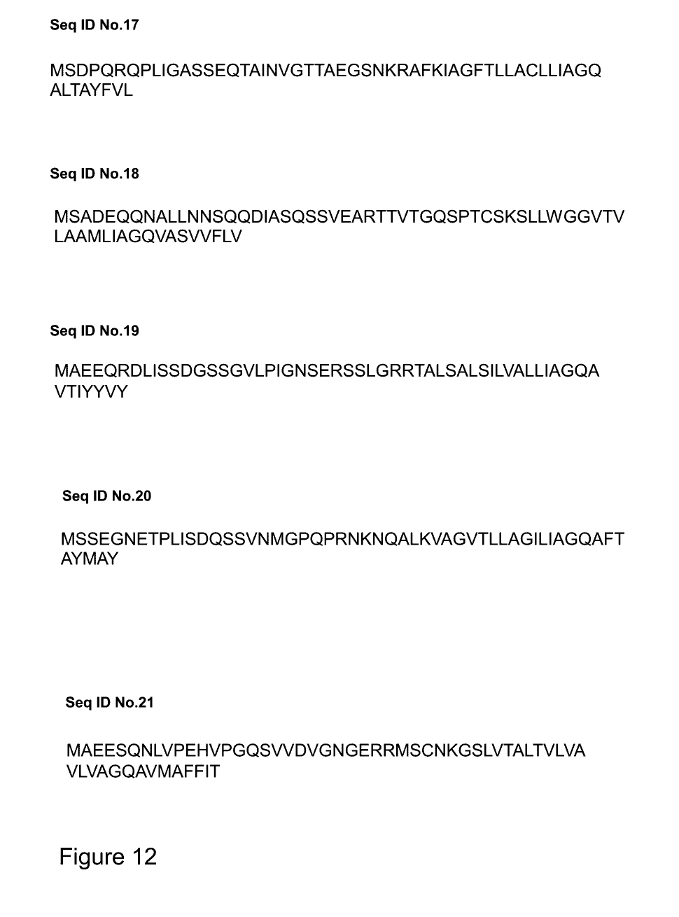

FIG. 12 shows the sequences of the truncated proteins used in the experiment shown in FIG. 11:

“trout” is a truncated version of “(1) rout”:

(SEQ ID NO 17)
MSDPQRQPLIGASSEQTAINVGTTAEGSNKRAFKIAGFTLLACLLIAG
QALTAYFVL,

“shark” is a truncated version of “(fl)shark”:

(SEQ ID NO 18)
MSADEQQNALLNNSQQDIASQSSVEARTTVTGQSPTCSKSLLWGGV
TVLAAMLIAGQVASVVFLV,

“chicken” is a truncated version of Seq ID No.2 in FIG. 9a:

(SEQ ID NO: 19)
MAEEQRDLISSDGSSGVLPIGNSERSSLGRRTALSALSILVALLIAGQ
AVTIYYVY,

“zebrafish” is a truncated version of Seq ID No.5 in FIG. 9b:

(SEQ ID NO: 20)
MSSEGNETPLISDQSSVNMGPQPRNKNQALKVAGVTLLAGILIAGQA
FTAYMAY,

“frog” is a truncated version of Seq ID No.8 in FIG. 9c:

(SEQ ID NO: 21)
MAEESQNLVPEHVPGQSVVDVGNGERRMSCNKGSLVTALTVLVAVL
VAGQAVMAFFIT.

### EXAMPLE 1

Design of li-ME-TRAP Fusion Protein

The ME-TRAP antigen construct comprises a human codon-optimized multi-epitope string (ME) fused to the native P. falciparum T9/96 strain cDNA sequence encoding TRAP (thrombospondin-related adhesive protein). Also known as sporozoite surface protein 2, TRAP is a type la membrane protein with a predicted N-terminal signal peptide, a large ectodomain, a transmembrane domain, and a short cytoplasmic C-terminal domain. This topology is compatible with fusion of the N-terminus of ME-TRAP to the C-terminus of Ii, since the latter is a type II membrane protein with a short cytoplasmic N-terminal domain. In order to prevent signal peptidase cleavage of the TRAP signal peptide, which (if it were to occur) would be predicted to result in hydrolysis of the peptide bond linking the antigen to li, nucleotides 1-75 of the TRAP open reading frame (ORF), which encodes a predicted signal peptide, were deleted from ME-TRAP in the versions fused to li. A mixture of gene synthesis and conventional cloning was used to make in-frame fusions of this modified ORF to synthetic ORFs (optimized to human codon usage) encoding fragments (shown in FIG. 2) of the human (NCBI RefSeq NP_004346.1; isoform b (FIG. 1 and SEQ ID NO.1)) or non-human li proteins, respectively.

### EXAMPLE 2

Construction of recombinant adenovirus vectors and recombinant MVA The above chimeric ORFs encoding ME-TRAP fused to fragments of human li (li-ME-TRAP) were sub-cloned into a transgene expression cassette comprising a modified human cytomegalovirus major immediate early promoter (CMV promoter) with tetracycline operator (TetO) sites. The cassettes were inserted into the E1 locus of an E1/E3-deleted and E4-modified genomic clone of a species E simian adenovirus such as ChAdOx1 and/or ChAd63, using site-specific recombination, The pre-existing comparator construct, ChAd.ME-TRAP, lacks TetO sites in the CMV promoter (which enable repression of transgene expression during viral production in 293 cells expressing the tetracycline repressor (TetR) protein), and was generated by recombination in BJ5183 cells, but is otherwise identical. The viruses were then rescued and propagated in 293 or 293-TetR cells, purified by CsCI gradient ultracentrifugation and titred as previously described. Doses for vaccination were based on infectious units (iu), since these, and not viral particles (vp), determine immunogenicity. ChAd particle-to-infectious-unit (P:l) ratios were in the range 50-120.

The ORF encoding hli-ME-TRAP variants were sub-cloned into a orthopoxviral expression plasmid to place it under control of the vaccinia virus p7.5 promoter and the cassette was introduced into the thymidine kinase (TK) locus of MVA by recombination in transfected and infected chick embryo fibroblast (CEF) cells followed by transient-dominant selection with a GFP marker gene. The resulting viral recombinant was plaque-purified and amplified in CEFs, purified over sucrose cushions and titred twice in duplicate in CEFs by an immunostained plaque assay, according to standard methods. The identity and purity of the isolate was verified by PCR. The comparator virus, MVA.ME-TRAP, has a LacZ marker gene, but is otherwise identical in design and was purified and titred similarly.

### EXAMPLE 3

Animals and Immunizations

Female C57BL/6J, Balb/c or CD-1(ICR) mice aged at least 6 weeks (Harlan, UK) were given intramuscular (i,m.) immunizations into the musculus tibialis with a total volume of 50 μl of vaccine diluted in endotoxin-free PBS using a 29G 0.5 ml insulin syringe (BD).

### EXAMPLE 4

### Immune Responses

Methods

Antigens for in vitro Re-Stimulation

For murine studies, cellular immune responses to TRAP were measured using in vitro restimulation with a single pool of synthetic peptides (20-mers overlapping by 10) spanning the entire TRAP sequence. Responses to ME were measured using the Plasmodium berghei circumsporozoite dominant H-2K^{d} restricted epitope Pb9 (SYIPSAEKI).

Intracellular Cytokine Staining (ICS)

Mouse splenocytes were treated with ACK to lyse erythrocytes prior to stimulation at 37° C. for 6 hours with 2 μg/ml of TRAP peptide pool with 1 μg/ml Golgi-Plug (BD). Following surface labelling with anti-CD4-e450 and anti-CD8-PerCPCy5.5 (all eBioscience) and staining with fixable Live/Dead Aqua

(Invitrogen), cells were fixed with neutral buffered formalin solution containing 4% formaldehyde (Sigma) for 5 minutes at 4° C., prior to intracellular staining with anti-TNF-a Alexa488, anti-IL-2-PE and anti-IFN-γAlexa647 (eBioscience) antibodies diluted in Perm-Wash buffer (BD).

Typically, antigen specific cells were identified by gating cells based on live cells, size, doublet negative and either CD4⁺CD8⁻ or CD4⁻CD8⁺. Statistical analysis was performed using Prism v5.0c (Graphpad).

Antibody Responses

Antibody response to TRAP were measured using a luciferase immunoprecipitation system (LIPS). The assay is based on binding of immobilised antibodies to a fusion protein of TRAP and Renilla luciferase (rLuc). Briefly, serum samples were incubated for 1 hour with a cell lysate from 293 cells transfected with a TRAP-rLuc expression plasmid, prior to incubation with Protein A/G UltraLink Resin beads (ThermoScientific) in MultiScreen HTS membrane Barex plates (Millipore) for 1 hour. Unbound lysate and antibodies were removed by washing the plates prior to quantification of bound rLuc activity using Renilla luciferase assay system (Promega) and a Varioskan Flash luminometer (Thermo). Antibody levels are expressed as log₁₀ luminescence units.

Results

To determine the ability of truncated variants to increase the response to ME-TRAP, Balb/c mice were vaccinated with 10⁷ iu of ChAd.ME-TRAP or ChAd.li-ME-TRAP fusions and two weeks later spleens harvested and cells restimulated with the Pb9 peptide (contained within the ME string) for 6 hours prior to staining for antigen specific cytokine production. Vaccination of mice with all li variants was shown to increase both CD4⁺ and CD8⁺ T cell responses compared to the control ME-TRAP, with the two shortest variants, 72aa and 92aa, demonstrating the greatest enhancement of the CD8 ⁺ T cell response (FIG. 3). Subsequently, C57BL/6 mice vaccinated with 10⁷ iu of control ChAd virus (ME-TRAP) or ChAd.hli-ME-TRAP (squares) or ChAd.72aa-ME-TRAP and 2 weeks later spleens harvested to measure TRAP specific response by intracellular cytokine staining (ICS) (FIG. 4). Vaccination with either variant of the li chain fusions induced a significant increase in the CD8⁺ T cell response, with the truncated 72aa hli chain shown to induce a significantly higher response than the full length li variant leading to an overall 15-fold increase compared to the control ME-TRAP vaccine.

To determine the ability of the truncated li to increase the immune response in a ChAd-MVA prime boost regimen, C57BL/6J mice were vaccinated with 10⁶ iu of the relevant ChAd vaccine and boosted 8 weeks later with 10⁶ PFU of the relevant MVA vaccine (FIG. 5). Vaccination with vaccines expressing the full-length li variant fused to ME-TRAP significantly increased the TRAP specific IFN-γ response compared to the control ME-TRAP vaccine, leading to an overall 26 fold increase in the response. Vaccination with the 72aa-li-ME-TRAP fusion induced an even higher CD8 ⁺ T cell response compared to the full-length human li variant, leading to overall 40-fold increase in the response compared to the control ME-TRAP vaccine. The effect of vaccinating with the 72aa li-ME-TRAP fusion expressed in either the ChAd, MVA, or both vaccines was subsequently assessed (FIG. 6). A small increase was observed in mice vaccinated with the ChAd.72aa li-ME-TRAP MVA.ME-TRAP regimen, with the greatest enhancement of the response observed when both the ChAd and MVA vaccines expressed the 72aa-li-ME-TRAP fusion. Vaccination with 72aahli-ME-TRAP was also shown to increase the level of TRAP specific antibodies after administration of ChAd vaccine, or ChAd-MVA prime-boost vaccination regimen (FIG. 7).

### EXAMPLE 5

### In vitro Antigen Expression

Methods

10⁶ A549 cells were seeded into 6-well plate containing a 22mm glass-cover slips and rested overnight. 3 μg of DNA plasmids expressing li variant-ME-TRAP fusion was incubated with lipofectamine 2000 according to the manufacturers instructions in Opti-MEM medium prior to addition to A549 cells. 24 hours later media was removed, cells were washed with PBS prior to fixation with 4% paraformaldehyde solution, quenching with 50 mM NH₄Cl and permeabilisation with 0.2% Triton X-100. Cells were stained with mouse anti-TRAP polyclonal serum for 1 hour at room temperature prior to washing in PBS and addition of secondary anti-mouse Alexa-488 (lnvitrogen) antibody.

Results

To investigate the ability of variant li sequences to increase and localise expression of ME-TRAP to intracellular compartments, A549 cells were transfected with DNA plasmids expression the variant-ME-TRAP fusions and stained for expression of TRAP by immunohistochemistry (FIG. 8). Fusion to to the truncated human li chain altered the expression pattern such that TRAP expression was localised intracellularly around the nucleus. This pattern of expression was also observed by fusion to either chicken, trout or shark li chains. These findings suggest that this rapid in vitro assay may be used to help identify truncations and variants of li that may be used as fusion partners to enhance the immune response to antigens. The findings furthermore indicate that variant li sequences from non-human species may be used to enhance antigen immunogenicity as alternatives to human li sequences,

The invention is not limited by any of the specific examples described herein, and the skilled person will appreciate the full range of alternatives available for each feature, each of which is intended to be covered, the invention being limited only by the claims. The contents of references provided throughout are incorporated herein in their entirety.

1. A nucleic acid construct encoding a protein fusion between an antigen and a invariant chain molecule, said invariant chain molecule consisting of a fragment of SEQ ID NO: 1, a variant of SEQ ID NO: 1, a variant of a fragment of SEQ ID No: 1 or the peptide of SEQ ID NO.1, the said variants having at least 85% sequence identity with the corresponding portion of SEQ ID NO.1, wherein the invariant chain molecule produces an enhanced CD4⁺ and/or CD8⁺ and/or antibody immune response against the antigen upon immunisation with the construct compared to the CD4⁺ and/or CD8⁺ and/or antibody immune response obtained by immunisation with a control construct encoding the antigen not fused to the invariant chain molecule.

2. A nucleic acid construct of claim 1 wherein the said invariant chain molecule consists of a fragment of SEQ ID NO.1 having:
(i) an N-terminus at any of positions 1 to 26 and a C-terminus at any of positions 72 to 87 of SEQ ID NO.1;
(ii) an N-terminus at positions 27 and a C-terminus at any of positions 72 to 75 or 77 to 87 of SEQ ID NO.1;
(iii) an N-terminus at positions 28 and a C-terminus at any of positions 72 to 74 or 78 to 87 of SEQ ID NO.1;
(iv) an N-terminus at positions 29 and a C-terminus at any of positions 72 to 73 or 79 to 87 of SEQ ID NO.1;
(v) an N-terminus at positions 30 and a C-terminus at any of positions 72 or 80 to 87 of SEQ ID NO.1;
(vi) an N-terminus at positions 31 and a C-terminus at any of positions 81 to 87 of SEQ ID NO.1;
(vii) an N-terminus at positions 32 and a C-terminus at any of positions 72 or 82 to 87 of SEQ ID NO.1;
(viii) an N-terminus at positions 33 and a C-terminus at any of positions 72 to 73 or 79 or 83 to 87 of SEQ ID NO.1;
(ix) an N-terminus at positions 34 and a C-terminus at any of positions 72 to 73 or 84 to 87 of SEQ ID NO.1;
(x) an N-terminus at positions 35 and a C-terminus at any of positions 72 or 85 to 87 of SEQ ID NO.1;
(xi) an N-terminus at positions 36 and a C-terminus at any of positions 86 to 87 of SEQ ID NO.1;
(xii) an N-terminus at positions 37 and a C-terminus at any of positions 72 or 87 of SEQ ID NO.1;
(xiii) an N-terminus at positions 38 and a C-terminus at any of positions 72 to 73 or 84 of SEQ ID NO.1;
(xiv) an N-terminus at positions 39 and a C-terminus at any of positions 72 to 74 or 78 of SEQ ID NO.1;
(xv) an N-terminus at positions 40 and a C-terminus at any of positions 72 to 75 or 77 of SEQ ID NO.1;
(xvi) an N-terminus at positions 41 and a C-terminus at any of positions 72 to 76 of SEQ ID NO.1;
(xvii) an N-terminus at positions 42 and a C-terminus at any of positions 72 to 77 of SEQ ID NO.1;
(xviii) an N-terminus at positions 43 and a C-terminus at any of positions 72 to 78 of SEQ ID NO.1;
(xix) an N-terminus at positions 44 and a C-terminus at any of positions 72 to 79 or 83 of SEQ ID NO.1;
(xx) an N-terminus at positions 45 and a C-terminus at any of positions 72 to 80 or 82 of SEQ ID NO.1;
(xxi) an N-terminus at positions 46 and a C-terminus at any of positions 72 to 81of SEQ ID NO.1;
(xxii) an N-terminus at positions 47 and a C-terminus at any of positions 72 to 82 of SEQ ID NO.1; or (xxiii) an N-terminus at any of positions 1 to 47 and a C-terminus at any of positions 97 or 98 of SEQ ID NO.1.

3. A nucleic acid construct of claim 1 wherein said invariant chain molecule consists of:
(i) amino acids 1 to 72 of SEQ ID NO.1;
(ii) amino acids 47 to 98 of SEQ ID NO.1;
(iii) amino acids 47 to 72 of SEQ ID NO.1;
(iv) amino acids 16 to 98 of SEQ ID NO.1; or
(v) amino acids 16 to 72 of SEQ ID NO.1

4. A nucleic acid construct of any preceding claim wherein the nucleic acid encoding the invariant chain molecule is replaced by a nucleic acid encoding a fragment of the invariant chain from a non-human species, such that the nucleic acid construct encodes a protein fusion between the antigen and a non-human invariant chain molecule, the said fragment of the invariant chain from a non-human species comprising the transmembrane domain of the full length invariant chain from that species.

5. A nucleic acid construct of claim 4 wherein the encoded non-human invariant chain molecule is derived from the invariant chain from chicken, quail, trout, zebrafish, carp, frog, grouper, shark, mandarin fish or mallard.

6. A nucleic acid construct of claim 5 wherein the encoded non-human invariant chain molecule is selected from:
(i) any of SEQ ID NO.s 2 to 12, 17 to 21; or
(ii) fragments of any of the sequences of SEQ ID NO.s 2 to 12, 17 to 21 comprising the transmembrane domain thereof; or
(iii) variants of the sequences in (i) or (ii) having at least 85% sequence identity therewith
the said fragments and variants producing an enhanced CD4⁺ and/or CD8⁺ and/or antibody immune response against the antigen upon immunisation with the construct compared to the CD4⁺ and/or CD8⁺ and/or antibody immune response obtained by immunisation with a control construct encoding the antigen not fused to the non-human invariant chain molecule.

7. A nucleic acid construct of any preceding claim wherein the invariant chain molecule or non-human invariant chain molecule is a variant, and wherein the degree of sequence identity between the invariant chain molecule or non-human invariant chain molecule and the variant thereof is at least 90%, optionally at least 95%, preferably at least 96%, 97%, 98% or 99%.

8. A nucleic acid construct of any preceding claim wherein the construct is a DNA vaccine.

9. A nucleic acid construct of any of claims 1 to 7 wherein the construct is RNA.

10. A nucleic acid construct of any of claims 1 to 7 wherein the construct is a viral vector, for example a viral vector derived from any of an adenovirus, such as chimpanzee adenoviruses, e.g. ChAdOx1 or ChAd63, a retrovirus, an alpha virus, an adeno-associated virus, a herpes virus, a vaccinia virus, a vaccinia derived virus e.g. MVA or NYVAC, a foamy virus, a cytomegalovirus, Semliki forest virus, a poxvirus, an avipox virus, e.g. canary pox or fowl pox, or an influenza virus, an RNA virus or a DNA virus.

11. A nucleic acid construct of any preceding claim wherein the components of the protein fusion are separated by a peptide linker.

12. A nucleic acid construct of any preceding claim wherein the antigen is derived from a pathogen.

13. A nucleic acid construct of any preceding claim wherein the antigen is associated with cancer, an autoimmune disease or an allergy.

14. A cell comprising a nucleic acid construct of any preceding claim, optionally wherein the cell is an antigen presenting cell.

15. A pharmaceutical composition, e.g. a vaccine, comprising a nucleic acid construct of any of claims 1 to 13 or a cell of claim 14 and a pharmaceutically acceptable excipient.

16. A nucleic acid construct of any of claims 1 to 13, a cell of claim 14 or a composition of claim 15 for use in therapy by immunisation.

17. A nucleic acid construct of any of claims 1 to 13, a cell of claim 14 or a composition of claim 15 for use in the prevention or treatment of an infectious disease such as malaria, an autoimmune disease, allergy or cancer by immunisation.

18. A method of eliciting an immune response comprising immunising a human or non-human animal with a nucleic acid construct of any of claims 1 to 13, a cell of claim 14 or a composition of claim 15.

19. A method of claim 18 for the treatment or prevention of disease in a human or non-human patient.

20. A non-therapeutic method of claim 18.

21. A nucleic acid construct, cell or composition for use of claim 16 or 17, or a method of any of claims 18 to 20, wherein the immunisation comprises a prime immunisation step and a boost immunisation step.

22. A nucleic acid construct, cell or composition for use of claim 21 wherein the prime immunisation is with a ChAd virus comprising the nucleic acid construct.

23. A nucleic acid construct, cell or composition for use of claim 21 or claim 22 wherein the boost immunisation is with a MVA virus comprising the nucleic acid construct.

24. A nucleic acid construct, cell or composition for use of any of claims 21 to 23 wherein the antigen is ME-TRAP.

25. A nucleic acid construct, cell or composition for use in the prevention or treatment of cancer of claim 17 wherein the antigen is a cancer-specific polypeptide selected from the group of: HPV derived viral oncogene E5, E6, E7 and L1; Survivin, BcI-XL, MCL-1, Rho-C, 5T4, PAP, PSA, STEAP, PSCA, PSMA, CEA and telomerase.

26. A nucleic acid construct, cell or vaccine for use of claim 17 wherein the autoimmune disease is selected from rheumatoid arthritis, systemic lupus erythematosus, multiple sclerosis, psoriasis and Crohn's disease.
